(19) United States
(12) Patent Application Publication
(10) Pub. No.: US 2018/0318399 A1
(43) Pub. Date: Nov. 8, 2018

(54) ANTIMICROBIAL COMPOSITIONS AND FORMULATIONS RELEASING HYDROGEN PEROXIDE

(71) Applicant: MATOKE HOLDINGS LIMITED, Abingdon, Oxfordshire (GB)

(72) Inventors: Sophie Constance COX, West Midland (GB); Thomas Jon HALL, West Midland (GB); Liam Michael GROVER, West Midland (GB); Rami SALIB, Eastleigh (GB); Thomas PATTON, Rathrippon, Collooney, County Sligo (IE); James BRENNAN, Hazelwood County Sligo (IE); John Reginald BARRETT, Templemore County Tipperary (IE); Ian STAPLES, Abingdon, Oxfordshire (GB)

(21) Appl. No.: 15/745,795

(22) PCT Filed: Jul. 22, 2016
(86) PCT No.: PCT/GB2016/052258

§ 371 Date: Jan. 18, 2018

(30) Foreign Application Priority Data
Jul. 23, 2015 (GB) 1513045.3
Apr. 12, 2016 (GB) 1606249.9

(51) Int. Cl. A61K 38/44; A61K 9/00; A61K 47/44; A61K 47/24; A61K 9/06; A61P 31/22; A61P 31/04

(52) U.S. Cl. CPC A61K 38/443; C12Y 101/03004; A61K 9/0014; A61P 31/04; A61K 47/24; A61K 9/06; A61P 31/22; A61K 47/44

(57) ABSTRACT

Compositions for generating antimicrobial activity are described. The compositions comprise: a first phase; a second phase; an enzyme that is able to convert a substrate to release hydrogen peroxide; and a substance that includes a substrate for the enzyme, wherein the first phase and the second phase are immiscible. The compositions may be formulated as colloids, suspensions or emulsions, especially as creams or sprays. Methods of making the compositions are described, as well as their use for the treatment of antimicrobial infections.

This invention relates to antimicrobial compositions and formulations, particularly colloids, suspensions or emulsions. The compositions and formulations may be for topical application for the treatment of antimicrobial infections, such as viral, bacterial, or fungal infections.

Cold sores are small blisters that develop on the lips or around the mouth. They are caused by the herpes simplex virus (HSV) and usually clear up without treatment within 7 to 10 days. Cold sores often start with a tingling, itching or burning sensation around the mouth. Small fluid-filled sores will then appear, most commonly on the edges of the lower lip. Topical products, such as creams or gels, can be used to treat cold sores. Many are prescription medicines that may slightly shorten the duration of cold sores, usually by just 1 to 2 days.

Genital herpes is a common infection caused by the herpes simplex virus (HSV). It causes painful blisters on the genitals and the surrounding areas. Herpes can be treated with anti-viral agents. However, these can cause side effects, such as nausea and headaches.

There remains a need for topical formulations that provide effective treatment of HSV and other viral or microbial infections.

The Applicant has found that compositions that are able to release hydrogen peroxide at the site of a microbial infection are particularly effective at preventing or inhibiting the infection.

Surgihoney™ is a chemical engineered honey that has the ability to deliver variable and sustained doses of reactive oxygen species (ROS). Studies in vitro and in vivo have demonstrated Surgihoney's efficacy in eradication of infection. This has included drug resistant strains, such as methicillin-resistant S. aureus (MRSA) and vanomycin-resistant Enterococcus faecium (Dryden, M., Lockyer, G., Saeed, K., & Cooke, J. (2014). Engineered Honey: In Vitro Antimicrobial Activity of a Novel Topical Wound Care Treatment. Journal of Global Antimicrobial Resistance, 2, 168-172). It was also shown to be effective against fungi and prevented or reduced the seeding of biofilms (Dryden, M., Halstead, F., & Cooke, J. (2015). Engineered Honey to Manage Bacterial Bioburden and Biofilm in Chronic Wounds. EWMA Free Paper Session: Infection and Antimicrobials). Chemically engineered honeys, such as honey with added glucose oxidase, are disclosed in WO 2015/166197 A1.

At present, Surgihoney™ is available in a sachet form for topical administration. However, administration of Surgihoney™ in this form may be inconvenient, make application of a controlled dose difficult and may be clinically non-optimal.

According to the invention there is provided a composition for generating antimicrobial activity, which comprises: a lipophilic phase; an aqueous phase; an enzyme that is able to convert a substrate to release hydrogen peroxide; and a substance that includes a substrate for the enzyme. A composition of the invention may be in the form of a colloid or a suspension.

The term “colloid” is used herein to refer to a homogeneous non-crystalline substance consisting of large molecules or ultramicroscopic particles of one substance dispersed through a second substance. Colloids include gels, sols, and emulsions. The particles do not settle, and cannot be separated out by ordinary filtering or centrifuging like those in a suspension.

The term “suspension” is used herein to refer to a mixture in which small particles of a substance are dispersed throughout a liquid. If a suspension is left undisturbed, the particles are likely to settle to the bottom. The particles in a suspension are larger than those in either a colloid or a solution.

A composition of the invention may be in the form of an emulsion. The term “emulsion” is used herein to refer to a fine dispersion of minute droplets of one liquid in another in which it is not soluble or miscible. An emulsion of the invention may be an oil and water emulsion, in particular an oil-in-water emulsion, or a water-in-oil emulsion. The composition may be a micro-emulsion.

Compositions of the invention may comprise a first phase (or first liquid, or first component) and a second phase (or second liquid, or second component), an enzyme that is able to convert a substrate to release hydrogen peroxide; and a substance that includes a substrate for the enzyme. The first phase and the second phase may be immiscible. For example, the first phase may be less polar than the second phase. The first phase may be a non-polar phase such as a lipophilic phase or a hydrophobic phase e.g. an oil. The second phase may be a polar phase, such as an aqueous phase. The second phase may comprise a non-aqueous solvent. Droplets or micelles of the second phase may be dispersed within the first phase.

The second phase may comprise water and/or non-aqueous solvent. The enzyme and the substance of the composition may be dissolved in the water and/or non-aqueous solvent.

It is conceivable that, in some embodiments, the second phase may not comprise water or may comprise substantially no water. In such circumstances, the second phase may be described as non-aqueous. For example, the enzyme and the substance comprising a substrate for the enzyme may be dissolved in a non-aqueous solvent. The non-aqueous solvent may be immiscible with respect to the first phase e.g. lipophilic phase.

In some embodiments, the enzyme that is able to convert a substrate to release hydrogen peroxide and the substance that includes a substrate for the enzyme may be contained within micelles dispersed within the first phase, e.g. lipophilic phase.

In some compositions, the composition may be in the form of a double emulsion. For example, droplets containing the enzyme that is able to convert a substrate to release hydrogen peroxide and the substance that includes a substrate for the enzyme may be dispersed within globules of a lipophilic phase (e.g. oil globules) and globules may be dispersed within an aqueous phase. Such a double emulsion may be termed a water-in-oil-in-water type (W/O/W) emulsion.

A composition of the invention may further comprise an emulsifying agent (or emulsifier). Emulsions can be stabilized by adsorption of surface active agents (emulsifying agents) at the emulsion interface. Emulsifying agents lower the interfacial tension to maintain the droplets in a dispersed state. An emulsifying agent has a hydrophilic part and a lipophilic part. It is possible to calculate the relative quantities of an emulsifying agent(s) necessary to produce the most physically stable emulsions for a particular formulation with water combination. This approach is called the hydrophilic-lipophilic balance (HLB) method (“The HLB SYSTEM a time-saving guide to emulsifier selection” ICI Americas Inc., Wlimington, Del. 19897, 1976, revised 1980). Each emulsifying agent is allocated an HLB number representing the relative properties of the lipophilic and hydrophilic parts of the molecule. High numbers (up to a theoretical number of 20), indicates an emulsifying agent exhibiting mainly hydrophilic or polar properties, whereas low numbers represent lipophilic or non-polar characteristics. According to the HLB System, all fats and oils have a Required HLB. Emulsions with optimal performance can be yielded by matching the HLB requirement with the emulsifying agent's HLB value. For an oil-in-water emulsion, the more polar the oil phase the more polar the emulsifying agent(s) must be. For example, to emulsify Soybean Oil, which has a Required HLB of 7, according to the HLB system, it would be necessary to use an emulsifying agent, or blend of emulsifying agents, with an HLB of 7±1. The HLB of emulsifying agents can be calculated or determined through trial and error.

Thus, the lipophilic phase of a composition of the invention may require an emulsifying agent of a particular HLB number in order to ensure a stable product. The lipophilic phase of a composition of the invention may comprise an oil or a wax. Examples of oils and waxes (by their International Nomenclature of Cosmetic Ingredients, INCI, name) for use in a lipophilic phase of a composition of the invention (with their respective Required HLBs) include the following:

Aleurites Moluccana Seed Oil [7]
Almond Oil NF [6]
Anhydrous Lanolin USP [10]
Apricot Kernel Oil [7]
Avocado (Persea Gratissima) Oil [7]
Babassu Oil [8]
Beeswax [12]
Borage (Borago Officinalis) Seed Oil [7]
Brazil Nut Oil [8]
C12-15 Alkyl Benzoate [13]
Cannabis Sativa Seed Oil [7]
Canola Oil [7]
Caprylic/Capric Triglyceride [5]
Carrot (Daucus Carota Sativa) Seed Oil [6]
Castor (Ricinus Communis) Oil [14]
Ceresin [8]
Cetearyl Alcohol [15.5]
Cetyl Alcohol [15.5]
Cetyl Esters [10]
Cetyl Palmitate [10]
Coconut Oil [8]
Daucus Carota Sativa (Carrot) Root Extract [6]
Diisopropyl Adipate [9]
Dimethicone [5]
Dog Rose (Rosa Canina) Hips Oil [7]
Emu Oil [8]
Evening Primrose Oil [7]
Grape (Vitis Vinifera) Seed Oil [7]
Hybrid Safflower (Carthamus Tinctorius) Oil [9]
Isopropyl Myristate [11.5]
Isopropyl Palmitate [11.5]
Jojoba (Buxus Chinensis) Oil [6.5]
Lanolin [10]
Macadamia (Ternifolia) Nut Oil [7]
Mangifera Indica (Mango) Seed Butter [8]
Mineral Oil [10.5]
Myristyl Myristate [8.5]
Olive (Olea Europaea) Oil [7]
Oryza Sativa (Rice Bran) Oil [7]
Peanut Oil NF [6]
Petrolatum [7]
PPG-15 Stearyl Ether [7]
Retinyl Palmitate [6]
Safflower (Carthamus Tinctorius) Oil [8]
Sesame (Sesamum Indicum) Oil [7]
Shea Butter (Butyrospermum Parkii) [8]
Soybean (Glycine Soja) Oil [7]
Stearic Acid [15]
Stearyl Alcohol [15.5]
Sunflower (Helianthus Annus) Oil [7]
Sweet Almond (Prunus Amygdalus
Dulcis) Oil [7]
Theobroma Cacao (Cocoa) Seed Butter [6]
Tocopherol [6]

In some embodiments, the lipophilic phase of a composition of the invention comprises a beeswax.

In some embodiments, the lipophilic phase is an oil. In some embodiments, the oil is selected from olive oil, corn oil, vegetable oil, sunflower oil or paraffin oil. In a preferred embodiment, the oil may be olive oil. In another preferred embodiment, the oil may be paraffin oil.

Water-in-oil emulsifying agents for use in compositions of the invention may have an HLB value in the range 3-6. Oil-in-water emulsifying agents for use in compositions of the invention may have an HLB value in the range 8-18. Examples of emulsifying agents (by their INCI name) for use in compositions of the invention (with their HLB numbers) include the following:

Calcium Stearoyl Lactylate [HLB = 5.1 ± 1]
Ceteareth-20 [HLB = 15.2 ± 1]
Cetearyl Glucoside [HLB = 11 ± 1]
Ceteth-10 [HLB = 12.9 ± 1]
Ceteth-2 [HLB = 5.3 ± 1]
Ceteth-20 [HLB = 15.7 ± 1]
Cocamide MEA [HLB = 13.5 ± 1]
Glyceryl Laurate [HLB = 5.2 ± 1]
Glyceryl Stearate [HLB = 3.8 ± 1]
Glyceryl Stearate (and) PEG-100 Stearate
[HLB = 11 ± 1]
Glyceryl Stearate SE [HLB = 5.8 ± 1]
Glycol Distearate [HLB = 1 ± 1]
Glycol Stearate [HLB = 2.9 ± 1]
Isoceteth-20 [HLB = 15.7 ± 1]
Isosteareth-20 [HLB = 15 ± 1]
Lauramide DEA [HLB = 15 ± 1]
Laureth-23 [HLB = 16.9 ± 1]
Laureth-4 [HLB = 9.7 ± 1]
Lecithin [HLB = 4 ± 1]
Lecithin [HLB = 9.7 ± 1]
Linoleamide DEA [HLB = 10 ± 1]
Methyl Glucose Sesquistearate
[HLB = 6.6 ± 1]
Oleth-10 [HLB = 12.4 ± 1]
Oleth-10/Polyoxyl 10 Oleyl Ether NF
[HLB = 12.4 ± 1]
Oleth-2 [HLB = 4.9 ± 1]
Oleth-20 [HLB = 12.4 ± 1]
Oleth-20 [HLB = 15.3 ± 1]
PEG-100 Stearate [HLB = 18.8 ± 1]
PEG-20 Almond Glycerides [HLB = 10 ± 1]
PEG-20 Methyl Glucose Sesquistearate
[HLB = 15 ± 1]
PEG-25 Hydrogenated Castor Oil
[HLB = 10.8 ± 1]
PEG-30 Dipolyhydroxystearate [HLB = 5.5 ± 1]
PEG-4 Dilaurate [HLB = 6 ± 1]
PEG-40 Sorbitan Peroleate [HLB = 9 ± 1]
PEG-60 Almond Glycerides [HLB = 15 ± 1]
PEG-8 Laurate [HLB = 13 ± 1]
PEG-80 Sorbitan Laurate [HLB = 19.1 ± 1]
Polysorbate 20 [HLB = 16.7 ± 1]
Polysorbate 60 [HLB = 14.9 ± 1]
Polysorbate 80 [HLB = 15 ± 1]
Polysorbate 85 [HLB = 11 ± 1]
Sodium Stearoyl Lactylate [HLB = 8.3 ± 1]
Sorbitan Isostearate [HLB = 4.7 ± 1]
Sorbitan Laurate [HLB = 8.6 ± 1]
Sorbitan Oleate [HLB = 4.3 ± 1]
Sorbitan Sesquioleate [HLB = 3.7 ± 1]
Sorbitan Stearate [HLB = 4.7 ± 1]
Sorbitan Stearate (and) Sucrose Cocoate
[HLB = 6 ± 1]
Sorbitan Trioleate [HLB = 1.8 ± 1]
Stearamide MEA [HLB = 11 ± 1]
Steareth-2 [HLB = 4.9 ± 1]
Steareth-21 [HLB = 15.5 ± 1]

In some embodiments, an emulsifying agent of a composition of the invention comprises a lecithin.

Emulsifying agents include ionic or non-ionic surfactants, and lipophilic fatty amphiles (for example, fatty alcohols or fatty acids). Non-ionic surfactants may be preferred since they may be less irritating to skin that anionic or cationic surfactants.

Other examples of suitable emulsifying agents include: Surfactants: Sodium lauryl sulphate, Cetrimide, Cetomacrogol 1000, PEG 1000 monostearate, Triethanolamine stearate, Sodium stearate; Fatty amphiphiles: Cetostearyl alcohol, Cetyl alcohol, Stearyl alcohol, Glyceryl monostearate, Stearic acid, Phosphatidylcholine.

Examples of commercial emulsifying waxes include: Emulsifying wax BP (Cetostearyl alcohol, sodium lauryl sulphate), Emulsifying wax USNF (Cetyl alcohol, polysorbate), Cationic emulsifying wax BPC (Cetostearyl alcohol, cetrimide), Glyceryl monostearate S. E. (Glyceryl monostearate, sodium stearate), Cetomacrogol emulsifying wax BPC (Cetostearyl alcohol, cetomacrogol 1000), Polawax (Cetyl alcohol, non-ionic surfactant), Lecithin (Phosphatidylcholine, phosphatidylethanolamine, phosphatidylinositol, phosphatidic acid).

Surfactants for use in compositions of the invention may include one or more of TWEEN (e.g. TWEEN 80), SPAN (e.g. SPAN 80), Poloxamer (e.g. Poloxamer 407) and Polyglycerol polyricinoleate (PGPR). A preferred surfactant may be Poloxamer, such as Poloxamer 407. Another preferred surfactant may be PGPR.

Surfactants may include a surfactant polymer, or co-polymer. For example, a suitable surfactant may be a triblock copolymer consisting of a central hydrophobic block flanked by two hydrophilic blocks.

According to the invention, there is provided a composition for generating antimicrobial activity, which comprises: an oil; an emulsifier; an enzyme that is able to convert a substrate to release hydrogen peroxide; and a substance that includes a substrate for the enzyme.

Compositions of the invention may comprise non-aqueous solvent. The non-aqueous solvent may be a polar solvent, such as a solvent with a dielectric constant of greater than 15. The non-aqueous solvent may be an organic solvent. For example, the solvent may be, or may comprise, glycerol, dimethylsulphoxide, propylene glycol or polyethylene glycol. The non-aqueous solvent may be immiscible with respect to the first phase e.g. the lipophilic phase (such as oil).

In a preferred embodiment, the non-aqueous solvent may be, or comprise, glycerol.

In some embodiments, compositions of the invention may comprise micelles, preferably reverse micelles. Within each micelle may be the enzyme and the substance (which may comprise an unrefined natural substance, such as honey), and outside of the micelle may be the first phase, e.g. the lipophilic phase (such as oil). Within each reverse micelle there may also be water and/or non-aqueous solvent. Within each micelle there may not be sufficient water for the enzyme to convert the substrate.

Compositions of the invention may comprise further components which may assist in reducing coalescence. Coalescence describes the situation in which two or more droplets, or micelles, combine to form a single droplet, or micelle. In order to reduce or prevent, coalescence, the strength of the interfacial film, i.e. the interface between the lipophilic phase and the aqueous phase, may be strengthened. This may be achieved, for example, by increasing the surfactant concentration, including an amphiphilic polymer, and/or by adding an alcohol, such as an aliphatic alcohol with 5-7 carbon atoms.

Compositions of the invention may be suitable for topical application, in particular topical application to a human subject. A composition for topical application may be applied to body surfaces such as skin or mucous membranes. Compositions of the invention for topical application may be in the form, for example, of a cream, a lotion, or a lip balm.

The term “cream” is used herein to refer to a semi-solid emulsion of oil-in-water, or water-in-oil, for topical use. Oil-in-water (o/w) creams are composed of small droplets of oil dispersed in a continuous aqueous phase, and water-in-oil (w/o) creams are composed of small droplets of water dispersed in a continuous oily phase. Oil-in-water creams are less greasy and more easily washed off using water. Water-in-oil creams are more moisturising as they provide an oily barrier which reduces water loss from the outermost layer of the skin.

The term “cream” may also refer to a semi-solid emulsion in which droplets of a first phase are dispersed in a continuous second phase, or in which droplets of a second phase are dispersed in a continuous first phase. For example, the first phase may be less polar than the second phase. The first phase may be a non-polar phase such as a lipophilic phase or a hydrophobic phase e.g. an oil. The second phase may be a polar phase, such as an aqueous phase. The second phase may comprise a non-aqueous solvent. The second phase may comprise water and/or non-aqueous solvent. It is conceivable that, in some embodiments, the second phase may not comprise water or may comprise substantially no water. In such circumstances, the second phase may be described as non-aqueous. The non-aqueous solvent may be immiscible with respect to the first phase e.g. lipophilic phase.

The term “lotion” is used herein to refer to a liquid suspension or emulsion for topical application. A lotion may comprise finely powdered, insoluble solids held in suspension by suspending agents and/or surface-active agents, or an emulsion (particularly, an oil-in-water emulsion) stabilized by one or more surface-active agents. A lotion has lower viscosity than a cream.

The term “lip balm” is used herein the refer to a wax-like substance applied topically to the lips of the mouth to moisturize and relieve chapped or dry lips. Lip balm may include, for example, beeswax or carnauba wax, camphor, cetyl alcohol, lanolin, paraffin, and petrolatum, among other ingredients.

Advantageously, compositions of the invention may be sprayable. For example, this may assist in overcoming some of the difficulties in applying Surgihoney in its conventional form.

For example, in some circumstances, Surgihoney (which may be sticky and viscous) may be difficult to apply to an infection site. Consequently, compositions of the invention may be delivered to a patient using a spray device. The device may be a spraying or atomising device, such as a pump-action spray or an aerosol spray. The invention may thus provide a spraying device comprising a composition of the invention.

Compositions of the invention may be suitable for internal administration to a subject. For example, the composition may be suitable for administration into a subject's respiratory tract. Surgihoney, in its conventional form, may not be easily administered to a subject's respiratory tract.

Compositions of the invention may be administered to the respiratory tract using a nebuliser or an inhaler. Consequently, the invention may provide a nebuliser of inhaler comprising a composition of the invention.

A nebuliser is a device that converts liquid into aerosol droplets suitable for inhalation. Nebulisers use oxygen, compressed air or ultrasonic power to break up medication solutions and deliver a therapeutic dose of aerosol particles directly to the lungs. A wide variety of nebulisers is available. Nebulisers can be driven by compressed gas (jet nebuliser) or by an ultrasonically vibrating crystal (ultrasonic nebuliser).

In order to produce small enough particles from solution in 5-10 minutes, gas flow rates of at least 6 L/minute are usually necessary. Ultrasonic nebulisers use a rapidly vibrating piezoelectric crystal to produce aerosol particles. Ultrasonic nebuliser machines are often smaller and quieter.

Many nebulisers deliver only 10% of the prescribed drug dose to the lungs. Much of the drug is caught on the internal apparatus or wasted during exhalation. The efficiency of drug delivery depends on the type and volume of nebuliser chamber and the flow rate at which it is driven. Some chambers have reservoir and valve systems to increase efficiency of particle delivery during inspiration and reduce environmental losses during expiration. Breath-assisted open vent systems improve drug delivery but are dependent on the patient having an adequate expiratory flow. Face masks or mouthpieces may be used for administration of aerosol particles.

Nebulisers are used for the treatment of many respiratory diseases. Indications for nebuliser use include the management of exacerbations and long-term treatment of chronic obstructive pulmonary disease (COPD), management of cystic fibrosis, bronchiectasis, asthma, HIV/AIDS and symptomatic relief in palliative care.

Nebulised compositions of the invention may be used to prevent or treat a microbial infection, for example a microbial infection that comprises a biofilm, or a microbe that is capable of forming a biofilm, in a subject suffering from respiratory disease, such as COPD, cystic fibrosis, bronchiectasis, or asthma, or an HIV/AIDS-associated respiratory infection, or respiratory infection associated with terminal disease.

A composition of the invention may be used to prevent or treat a microbial infection that comprises a biofilm, or a microbe that is capable of forming a biofilm. The biofilm may comprise a biofilm-forming bacterium, fungus, or virus. The microbe that is capable of forming a biofilm may be a bacterium, fungus, or virus.

In some embodiments, a composition used to prevent or treat a microbial infection may exclude a nebulised composition of the invention.

The enzyme of a composition of the invention may be additional (i.e. added as a result of human intervention) to any enzyme activity able to convert the substrate to release hydrogen peroxide (referred to herein as “substrate conversion activity”) that may be present in the substance, i.e. the composition may comprise the substance and added enzyme. In some embodiments there may be no substrate conversion activity in the substance.

A composition of the invention may be a storage-stable composition which does not include sufficient free water to allow the enzyme to convert the substrate.

For example, in some embodiments, the enzyme and the substance comprising a substrate for the enzyme may be encapsulated or contained within micelles (such as reverse micelles), and within the micelles there may not be sufficient free water to allow the enzyme to convert the substrate. A non-aqueous solvent, may be present in the micelles.

Alternatively, compositions of the invention may be storage stable by virtue of the enzyme that is able to convert a substrate to release hydrogen peroxide and the substance that includes a substrate for the enzyme, being separate (or compartmentalised) from water in the composition. For example, the composition may be a double emulsion. Droplets containing the enzyme and the substance (but without sufficient free water to allow the enzyme to convert the substrate) may be dispersed within globules of oil, and the globules of oil may be dispersed within an aqueous phase (e.g. water).

In the presence of sufficient water, the enzyme of the storage-stable composition is able to convert the substrate and release hydrogen peroxide. Hydrogen peroxide is known to be effective against a wide variety of different microbes. Thus, antimicrobial activity is generated following dilution of a storage-stable composition of the invention.

If a storage-stable composition is used, this may be diluted by liquid present at the site of administration leading to release of hydrogen peroxide at the administration site.

Compositions of the invention that do not include water, or any free water, may provide particularly stable compositions of the invention, since the enzyme will not be able to convert the substrate to release hydrogen peroxide until the composition is contacted with sufficient amount of water.

Catalase is an enzyme that catalyses the decomposition of hydrogen peroxide to water and oxygen. The use of a substance that lacks catalase activity means that there is no variability in the amount of this activity between similar substances from different sources, or from different harvests from the same source. This reduces the variability in antimicrobial activity that can be generated from such substances. Alternatively, if the substance does include catalase activity, and it is not possible or desirable to inactivate the catalase activity in the substance prior to contacting the substance with the enzyme, then sufficient enzyme may be used such that the effect of catalase activity on the hydrogen peroxide that can be generated from the substance is reduced. This also reduces the variability in antimicrobial activity that can be generated from the substance. In some embodiments, the substance may lack catalase activity.

Catalase is present in many plants and animals. Catalase activity may be removed during processing or extraction of the substance, or inactivated before use of the substance in the composition. Catalase activity may be heat inactivated, for example by pasteurisation. A suitable temperature for heat inactivation of catalase activity is at least 60° C., 70° C., or 80° C., preferably for at least 2 minutes.

The term “storage-stable” is used herein to mean that the composition can be stored at ambient temperature for at least several days, suitably at least a week or at least one or two months, whilst retaining the ability to generate antimicrobial activity following dilution of the composition. The storage temperature may be below 37° C., preferably 20-25° C. Preferably compositions are stored away from exposure to light.

Hydrogen peroxide is generally unstable at ambient temperature. The lack of sufficient free water in a storage-stable composition of the invention prevents the enzyme converting the substrate to release hydrogen peroxide, and thus helps to maintain the stability of the composition for extended periods at ambient temperature. A storage-stable composition of the invention may include some water provided that there is not sufficient free water to allow the enzyme to convert the substrate. Suitable amounts of water will vary depending on the precise components of the composition. However, typically, a storage-stable composition of the invention comprises less than 20% total water content, for example, 10%-19%, water.

Hydrogen peroxide may be released for a sustained period following dilution of the composition, depending on the amount of substrate present in the composition, and the activity of the enzyme. It will be appreciated that the amount of substrate and/or the activity of enzyme in the composition may be selected to provide for release of a relatively high level of hydrogen peroxide for a short period, or for release of a lower level of hydrogen peroxide for a longer period, following dilution of the composition. Suitably the composition provides for sustained release of hydrogen peroxide for a period of at least twenty four hours, more preferably at least forty eight hours, following dilution of the composition. Suitably the composition provides for sustained release of hydrogen peroxide at a level of less than 2 mmol/litre for a period of at least twenty four hours, following dilution of the composition.

A composition of the invention may comprise sufficient enzyme and substrate to provide for sustained release of at least 0.1, 0.5, 1 or 1.5 mmol/litre hydrogen peroxide for a period of at least 24 hours, more preferably 48 hours.

It will be appreciated that there should be sufficient enzyme present in a storage-stable composition of the invention to convert the substrate and form hydrogen peroxide as needed following dilution of the composition.

In view of the importance of generation of hydrogen peroxide by storage-stable compositions of the invention in the presence of sufficient water, it will be appreciated that the compositions should not contain any added peroxidase.

In some embodiments the enzyme is a purified enzyme. The term “purified enzyme” is used herein to include an enzyme preparation in which the enzyme has been separated from at least some of the impurities originally present when the enzyme was produced. Preferably impurities that have been removed or reduced include those that would otherwise interfere with the ability of the enzyme to convert the substrate to release hydrogen peroxide.

It may not always be necessary or desirable that the purified enzyme is at a high level of purity provided that the enzyme is able to convert the substrate to release hydrogen peroxide. In some circumstances, it may be desirable to use a relatively crude enzyme preparation. Examples of suitable purity levels include at least 10%, 20%, 30%, 40%, 50%, 60%, 70%, 80%, or 90% pure.

It is preferred, however, that the amount of any catalase that may originally have been present when the enzyme was produced has been reduced. The enzyme may have been produced by recombinant or non-recombinant means, and may be a recombinant or non-recombinant enzyme. The enzyme may be purified from a microbial source, preferably from a non-genetically modified microbe.

The level of purity of the enzyme may be selected as appropriate depending on the intended use of the composition. For example, if the composition is intended for medical use, a medical grade or medical device grade of purity should be used.

Thus, there is provided according to the invention a storage-stable composition for generating antimicrobial activity, which comprises: a lipophilic phase; an aqueous phase; a purified enzyme that is able to convert a substrate to release hydrogen peroxide; and a substance that includes a substrate for the enzyme; wherein the composition does not include sufficient free water to allow the enzyme to convert the substrate.

According to the invention, there is provided a storage-stable composition for generating antimicrobial activity, comprising: a first phase; a second phase; an enzyme that is able to convert a substrate to release hydrogen peroxide; and a substance that includes a substrate for the enzyme, wherein the first phase and the second phase are immiscible, and wherein the composition does not comprise sufficient free water to allow the enzyme to convert the substrate.

According to the invention, there is provided a storage-stable composition for generating antimicrobial activity, which comprises: an oil; an emulsifier; an enzyme that is able to convert a substrate to release hydrogen peroxide; and a substance that includes a substrate for the enzyme, wherein the composition does not include sufficient free water to allow the enzyme to convert the substrate.

In some embodiments the enzyme is an oxidoreductase enzyme. Examples of oxidoreductase enzymes that can convert a substrate to release hydrogen peroxide include glucose oxidase, hexose oxidase, cholesterol oxidase, galactose oxidase, pyranose oxidase, choline oxidase, pyruvate oxidase, glycollate oxidase, and amioacid oxidase. The corresponding substrates for these oxidoreductase enzymes are D-glucose, hexose, cholesterol, D-galactose, pyranose, choline, pyruvate, glycollate and aminoacid, respectively.

A mixture of one or more oxidoreductase enzymes and one or more substrates for the oxidoreductase enzymes may be present in a composition of the invention.

The oxidoreductase enzyme may be glucose oxidase, and the substrate may be D-glucose.

The substance may be any substance that includes a substrate for the enzyme. In some embodiments the substance lacks catalase activity.

Thus, there is also provided according to the invention a storage-stable composition for generating antimicrobial activity, which comprises: a lipophilic phase; an aqueous phase; an enzyme that is able to convert a substrate to release hydrogen peroxide; and a substance that lacks catalase activity and that includes a substrate for the enzyme; wherein the composition does not include sufficient free water to allow the enzyme to convert the substrate.

There is also provided, according to the invention, a storage-stable composition for generating antimicrobial activity, comprising: a first phase; a second phase; an enzyme that is able to convert a substrate to release hydrogen peroxide; and a substance that lacks catalase activity and that includes a substrate for the enzyme, wherein the first phase and the second phase are immiscible, and wherein the composition does not comprise sufficient free water to allow the enzyme to convert the substrate.

There is also provided according to the invention a storage-stable composition for generating antimicrobial activity, which comprises: an oil; an enzyme that is able to convert a substrate to release hydrogen peroxide; and a substance that lacks catalase activity and that includes a substrate for the enzyme, and wherein the composition does not include sufficient free water to allow the enzyme to convert the substrate.

The substance may be an unrefined substance. The term “unrefined” is used herein to refer to substances that have not been processed into a pure form. Unrefined substances include substances that may have been concentrated, for example by drying or boiling.

The substance may include one or more substrates from a natural source (termed herein a “natural substance”). Examples of natural substances include substances from a plant source, including from sap, roots, nectar, flowers, seeds, fruit, leaves, or shoots. The substance may be an unrefined natural substance.

Suitably the substance comprises one or more of the following substrates: D-glucose, hexose, cholesterol, D-galactose, pyranose, choline, pyruvate, glycollate or amino acid.

The substance may be a sugar substance. The term “sugar substance” is used herein to mean any substance that includes one or more sugars. The term “sugar” is used herein to refer to a carbohydrate with the general formula Cₘ(H₂O)ₙ. Preferred sugars include monosaccharides, such as D-glucose, hexose, or D-galactose. The sugar substance may include one or more sugars from a natural source (termed herein a “natural sugar substance”). The natural sugar substance may be an unrefined natural sugar substance. The unrefined natural sugar substance may be (or be derived from) a natural sugar product. In some embodiments, the unrefined natural sugar product is a honey. In some embodiments, the honey is a honey that has been treated to remove or inactivate catalase activity.

As discussed above, the substance itself may preferably lack an enzyme activity that is able to convert the substrate to release hydrogen peroxide (referred to as “substrate conversion activity”). Absence of substrate conversion activity from the substance has the advantage that there is then no variability in the amount of this activity between similar substances from different sources, or from different harvests from the same source. This further reduces the variability in antimicrobial activity that can be generated from such substances. Substrate conversion activity is then provided only by the enzyme that is contacted with the substance, and so the amount of substrate conversion activity present in the composition can be controlled.

Substrate conversion activity may be removed during processing or extraction of the substance, or inactivated before use of the substance in a composition of the invention. Substrate conversion activity may be inactivated by heat inactivation, for example by pasteurisation. A suitable temperature for heat inactivation of substrate conversion activity is at least 80° C., preferably for at least two minutes. An advantage of heat inactivation is that both catalase activity and substrate conversion activity can be inactivated in a single heat inactivation step.

In some embodiments of the invention, the substance is a processed, extracted, or refined substance (i.e. a substance in which impurities or unwanted elements have been removed by processing). Preferably impurities that have been removed or reduced include those that would otherwise interfere with the ability of the enzyme to convert the substrate to release hydrogen peroxide.

In some embodiments of the invention, the substance comprises a purified substrate for the enzyme. The term “purified substrate” is used herein to include a substrate preparation in which the substrate has been separated from at least some of the impurities originally present when the substrate was obtained or produced. The purified substrate may be obtained from a natural source or may be synthetically produced. The purified substrate may be a processed, extracted, or refined substrate (i.e. a substrate in which impurities or unwanted elements have been removed by processing).

It may not always be necessary or desirable that the purified substrate is at a high level of purity provided that the enzyme is able to convert the substrate to release hydrogen peroxide. In some circumstances, it may be desirable to used a relatively crude substrate preparation. Examples of suitable purity levels include at least 10%, 20%, 30%, 40%, 50%, 60%, 70%, 80%, 90%, 95%, or 99% pure. However, in some embodiments, it may be desirable that the purified substrate is a medical grade, medical device grade, or pharmaceutical grade substrate.

In particular embodiments, the purified substrate is or comprises a purified sugar substance. The purified sugar substance may be obtained from a natural source (for example a processed, extracted, or refined natural sugar substance), or be synthetically produced. The purified sugar substance may be at least 10%, 20%, 30%, 40%, 50%, 60%, 70%, 80%, 90%, 95%, or 99% pure. The purified sugar substance may be a medical grade, medical device grade, or pharmaceutical grade sugar substance. The purified sugar substance may include one or more purified sugar substances, for example purified D-glucose, hexose, or D-galactose. For example the purified sugar substance may be medical grade, medical device grade, or pharmaceutical grade D-glucose, hexose, or D-galactose.

There is also provided according to the invention a composition for generating anti-microbial activity, wherein the composition comprises: a lipophilic phase; an aqueous phase; an enzyme that is able to convert a substrate to release hydrogen peroxide; and a substance that includes a purified substrate for the enzyme.

There is also provided, according to the invention, a composition for generating antimicrobial activity, comprising: a first phase; a second phase; an enzyme that is able to convert a substrate to release hydrogen peroxide; and a substance that includes a purified substrate for the enzyme, wherein the first phase and the second phase are immiscible.

There is also provided according to the invention a composition for generating anti-microbial activity, wherein the composition comprises: an oil; an enzyme that is able to convert a substrate to release hydrogen peroxide; and a substance that includes a purified substrate for the enzyme.

The composition may be a storage-stable composition for generating antimicrobial activity, which comprises: a lipophilic phase; an aqueous phase; an enzyme that is able to convert a substrate to release hydrogen peroxide; and a substance that includes a purified substrate for the enzyme; wherein the composition does not include sufficient free water to allow the enzyme to convert the substrate.

The composition may be a storage-stable composition for generating antimicrobial activity, comprising: a first phase; a second phase; an enzyme that is able to convert a substrate to release hydrogen peroxide; and a substance that includes a purified substrate for the enzyme, wherein the first phase and the second phase are immiscible, and wherein the composition does not comprise sufficient free water to allow the enzyme to convert the substrate.

The composition may be a storage-stable composition for generating antimicrobial activity, which comprises: an oil; an enzyme that is able to convert a substrate to release hydrogen peroxide; and a substance that includes a purified substrate for the enzyme, and wherein the composition does not include sufficient free water to allow the enzyme to convert the substrate.

The composition may be a storage-stable composition for generating antimicrobial activity, which comprises: a lipophilic phase; an aqueous phase; an enzyme that is able to convert a substrate to release hydrogen peroxide; and a substance that lacks catalase activity and that includes a purified substrate for the enzyme; wherein the composition does not include sufficient free water to allow the enzyme to convert the substrate.

The composition may be a storage-stable composition for generating antimicrobial activity, comprising: a first phase; a second phase; an enzyme that is able to convert a substrate to release hydrogen peroxide; and a substance that lacks catalase activity and that includes a substrate for the enzyme, wherein the first phase and the second phase are immiscible, and wherein the composition does not include sufficient free water to allow the enzyme to convert the substrate.

The composition may be a storage-stable composition for generating antimicrobial activity, which comprises: an oil; an enzyme that is able to convert a substrate to release hydrogen peroxide; and a substance that lacks catalase activity and that includes a purified substrate for the enzyme; wherein the composition does not include sufficient free water to allow the enzyme to convert the substrate.

In particular embodiments, the enzyme and the substrate are purified, for example purified glucose oxidase and purified D-glucose, suitably medical grade, medical device grade, or pharmaceutical grade glucose oxidase and D-glucose.

The ratio of the lipophilic phase to the aqueous phase, or the ratio of the first phase to the second phase, in a composition of the invention may be from 9:1 to 1:9, 8:1 to 1:8, 7:1 to 1:7, 6:1 to 1:6, 5:1 to 1:5, 4:1 to 1:4, 3:1 to 1:3, or 2:1 to 1:2 (v/v), for example from 4:1 to 1:4.

A composition of the invention may comprise 5-95%, 10-95%, 15-95%, 20-95%, 25-95%, 30-95%, 35-95%, 40-95%, 45-95%, 50-95%, 55-95%, 60-95%, 65-95%, 70-95%, 75-95%, 80-95%, 85-95%, or 90-95% (v/v) lipophilic phase, or first phase (including any emulsifying agent present).

Alternatively, a composition of the invention may comprise 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, or 5-10% (v/v) lipophilic phase, or first phase (including any emulsifying agent present).

A composition of the invention may comprise 5-95%, 10-95%, 15-95%, 20-95%, 25-95%, 30-95%, 35-95%, 40-95%, 45-95%, 50-95%, 55-95%, 60-95%, 65-95%, 70-95%, 75-95%, 80-95%, 85-95%, or 90-95% (v/v) aqueous phase, or second phase.

Alternatively, a composition of the invention may comprise 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, or 5-10% (v/v) aqueous phase, or second phase.

A composition of the invention may comprise 1-60%, 1-50%, 1-40%, 1-30%, 1-20%, or 1-10% (w/v) of the substance, for example a honey.

A composition of the invention may comprise 1-60%, 5-60%, 10-60%, 15-60%, 20-60%, 25-60%, 30-60%, 35-60%, 40-60%, 45-60%, or 50-60% (w/v) of the substance, for example a honey.

A composition of the invention may comprise 1-1500 units, 15-1500 units, 30-1500 units, 50-1500 units, 100-1500 units, 1-<685 units, 15-<685 units, 30-<685 units, 50-<685 units, 100-<685 units, 500-1000 units, 685-1000 units, or 100-500 units, of the enzyme, preferably glucose oxidase, per gram of the composition.

A composition of the invention may comprise no more than 85% water, for example no more than 80%, 70%, 60%, 50%, 40%, 30%, or 20% water, or less than 20% water, for example 10-19% water. A composition of the invention may comprise less than 20% (w/w). A composition of the invention may comprise less than 15% (w/w) water. A composition of the invention may comprise less than 12% (w/w) water.

A composition of the invention may comprise 10-60% (w/w) of non-aqueous solvent. In some embodiments, a composition of the invention may comprise 20-50% (w/w) of a non-aqueous solvent. In some embodiments, a composition of the invention may comprise 35-40% (w/w) of a non-aqueous solvent.

A composition of the invention may comprise 10-40% (w/w) of the first phase, e.g. lipophilic phase (such as oil). The composition may comprise 20-30% (w/w) of the first phase, e.g. lipophilic phase (such as oil).

A composition of the invention may comprise 1-10% (w/w) emulsifier. The composition may comprise 1-5% (w/w) emulsifier. The emulsifier is preferably a surfactant.

A composition of the invention may comprise 10-50% (w/w) of the substance which comprises a substrate for the enzyme. The composition may comprise 20-40% (w/w) of the substance. In some embodiments, the composition may comprise 25 to 35% (w/w) of the substance.

A composition of the invention may comprise 20-50% (w/w) of non-aqueous solvent, 20-30% (w/w) of the first phase e.g. lipophilic phase (such as oil), 1-5% (w/w) emulsifier and 20-40% (w/w) of the substance which comprises a substrate for the enzyme.

A composition of the invention may comprise 10-60% (w/w) of non-aqueous solvent, 10-40% (w/w) of the first phase e.g. lipophilic phase (such as oil), 1-10% (w/w) emulsifier and 10-50% (w/w) of the substance which comprises a substrate for the enzyme.

A composition of the invention may comprise 35-45% (w/w) of non-aqueous solvent, 20-30% (w/w) of the first phase e.g. lipophilic phase (such as oil), 1-5% (w/w) emulsifier and 25-35% (w/w) of the substance which comprises a substrate for the enzyme.

A composition of the invention may comprise 30-60% (v/v) solvent, such as a non-aqueous , polar solvent.

A composition of the invention may comprise 30-60% (v/v) first phase, such as a lipophilic phase (e.g. oil),

A composition of the invention may comprise 1-10% (v/v) emulsifier such e.g. surfactant.

A composition of the invention may comprise 30-70% or 40-60% (w/w) of the substance that comprises a substrate for the enzyme, for example honey.

The ratio of the first phase to the second phase in a composition of the invention may be ≤1:1 (v/v), for example 0.1-1:1 (v/v). In some embodiments, the ratio of the first phase to the second phase is <0.6:1 (v/v), for example 0.1-<0.6:1 (v/v). In some embodiments, the ratio of the first phase to the second phase is ≤0.4:1 (v/v), for example 0.1-0.4:1 (v/v).

The first phase in a composition of the invention may be present at less than 60% (v/v) of the composition. In some embodiments, the first phase is present at 10% to less than 60% (v/v) of the composition. In some embodiments, the first phase is present at 10% to less than 50% (v/v) of the composition. In some embodiments, the first phase is present at 10% to less than 40% (v/v) of the composition. In some embodiments, the first phase is present at 10% to less than 30% (v/v) of the composition. In some embodiments, the first phase is present at 10% to less than 25% (v/v) of the composition.

A composition of the invention may comprise an emulsifier. In some embodiments, the emulsifier is present at up to 25% (v/v) of the composition, for example 1-25% (v/v) of the composition, 5-25% (v/v) of the composition, or 10-25% (v/v) of the composition.

The ratio of the amount of the substance that includes a substrate for the enzyme to the volume of the second phase in a composition of the invention may be from 0.5:1 to 2:1, for example 1:1.

The amount of the substance that includes a substrate for the enzyme in a composition of the invention may be up to 70% (w/v) of the composition, for example 5-70% (w/v), 10-70% (w/v), 20-70% (w/v), or 30-70% (w/v), or up to 60% (w/v) of the composition, for example 5-60% (w/v), 10-60% (w/v), 20-60% (w/v), or 30-60% (w/v), of the composition.

A composition of the invention may be an emulsion. In particular embodiments, a composition of the invention is an emulsion that comprises reverse micelles. The reverse micelles may be formed by the second phase.

In some embodiments of a composition of the invention, the enzyme and the substance that includes a substrate for the enzyme is dissolved in the second phase.

In particular embodiments of the invention, the first phase is, or comprises paraffin oil.

In particular embodiments of the invention, the second phase is, or comprises glycerol.

In particular embodiments of the invention, the emulsifier is, or comprises Polyglycerol polyricinoleate (PGPR).

In particular embodiments of the invention, the enzyme that is able to convert a substrate to release hydrogen peroxide is, or comprises purified glucose oxidase, and the substance that includes a substrate for the enzyme is, or comprises honey.

In other particular embodiments of the invention, the enzyme that is able to convert a substrate to release hydrogen peroxide is, or comprises purified glucose oxidase, and the substance that includes a substrate for the enzyme is, or comprises purified glucose.

In some embodiments, a composition of the invention is a cream. Typically, the viscosity of an emulsion used as a cream will be higher than that of an emulsion used as a spray. A cream may be formed by including a viscosity-increasing agent, such as a thickener or gelling agent (for example a hydrocolloid) in the composition.

Hydrocolloids are a heterogeneous group of hydrophilic, long-chain polymers (polysaccharides or proteins) characterised by their ability to form viscous dispersions and/or gels when dispersed in water (Saha and Bhattacharya, J Food Sci Technol, 2010, 47(6):587-597). The extent of thickening varies with the type and nature of the hydrocolloid. Some provide low viscosities at a fairly high concentration, but most provide a high viscosity at a concentration below 1%. The viscosity of hydrocolloid dispersions arises predominantly from non-specific entanglement of conformationally disordered polymer chains. Hydrocolloids that can be used as thickening agents (referred to herein as hydrocolloid thickeners) include starch, modified starch, xanthan, galactomannans (such as guar gum, locust bean gum, and tara gum), gum Arabic or acacia gum, gum karaya, gum tragacanth, konjac maanan, and cellulose derivatives such as carboxymethyl cellulose, methyl cellulose, and hydroxypropylmethyl cellulose.

Some hydrocolloids are able to form gels, consisting of polymer molecules cross-linked to form an interconnected molecular network immersed in a liquid medium. A rheological definition of a gel is a viscoelastic system with a ‘storage modulus’ (G′) larger than the ‘loss modulus’ (G″) (de Vries 2004, Gums and stabilizers for the food industry, vol 12. RSC Publ, Oxford, pp 22-30). Hydrocolloids form gels by physical association of their polymer chains through hydrogen bonding, hydrophobic association, and cation-mediated cross-linking. Gelling-type hydrocolloids (or hydrocolloid gelling agents) include alginate, pectin, carrageenan, gelatin, gellan, agar, modified starch, methyl cellulose and hydroxypropylmethyl cellulose.

Gelation of hydrocolloids can occur by different mechanisms: ionotropic gelation, cold-set gelation and heat-set gelation (Burey et al. 2008, Crit Rev Food Sci Nutr 48:361-377). lonotropic gelation occurs via cross-linking of hydrocolloid chains with ions, typically a cation-mediated gelation process of negatively-charged polysaccharides. Examples of hydrocolloids that can form gels by ionotropic gelation include alginate, carrageenan and pectin. lonotropic gelation can be carried out by either diffusion setting or internal gelation. In cold-set gelation, hydrocolloid powders are dissolved in warm/boiling water to form a dispersion which forms a gel on cooling. Agar and gelatin form gels by this mechanism. Heat-set gels require the application of heat to gel (for example, curdlan, konjac glucomannan, methyl cellulose, starch and globular proteins).

Thus, in some embodiments, a composition of the invention comprises a viscosity-increasing agent, such as a thickener or gelling agent, for example a hydrocolloid. In particular embodiments, the hydrocolloid is, or comprises, a polysaccharide or a protein. The hydrocolloid may be a hydrocolloid thickener, such as starch, modified starch, xanthan, a galactomannan (such as guar gum, locust bean gum, and tara gum), gum Arabic or acacia gum, gum karaya, gum tragacanth, konjac maanan, or a cellulose derivative, such as carboxymethyl cellulose, methyl cellulose, or hydroxypropylmethyl cellulose.

In other embodiments, the hydrocolloid is, or comprises a cross-linked hydrocolloid, for example a cross-linked polysaccharide, such as cross-linked alginate, pectin, carrageenan, gelatin, gellan, agar, agarose, modified starch, or a cellulose derivative, such as methyl cellulose or hydroxypropylmethyl cellulose.

The hydrocolloid may be cross-linked by any suitable method, for example including the methods for gelation of hydrocolloids described above: ionotropic gelation, cold-set gelation and heat-set gelation. In particular embodiments, molecules of the hydrocolloid are cross-linked by cations (for example calcium ions) as a result of ionotropic gelation of a hydrocolloid gelling agent. Examples of hydrocolloid cross-linked by cations that may be present in a composition of the invention include alginate, carrageenan or pectin.

In particular embodiments, a composition of the invention includes cross-linked alginate, for example alginate cross-linked by calcium ions. Alginate can form gels without prior heating because sodium alginate is soluble in cold water.

Cross-linked alginate may be formed from sodium alginate and calcium ions (for example, provided by calcium chloride). In some embodiments, water may be used as solvent to dissociate the calcium ions. However, since this could potentially activate production of hydrogen peroxide by the enzyme and the substance that includes a substrate for the enzyme, and limit the stability of the composition, it may be preferred to use a non-aqueous solvent to dissociate the calcium ions, such as ethanol or acetic acid.

We have appreciated that glycerol may be used to bind free water. This property allows water to be used to dissolve the alginate, provided sufficient glycerol is present to prevent premature release of hydrogen peroxide from the enzyme and the substance that includes a substrate for the enzyme.

There is also provided according to the invention a method of making a composition of the invention, which comprises mixing a lipophilic component, an aqueous component, an enzyme that is able to convert a substrate to release hydrogen peroxide, and a substance that includes a substrate for the enzyme to form the composition.

There is also provided, according to the invention, a method of making a composition of the invention, comprising mixing a first component (or liquid of a first phase), a second component (or liquid of a second phase), an enzyme that is able to convert a substrate to release hydrogen peroxide, and a substance that includes a substrate for the enzyme to form the composition, wherein the first component (or liquid of the first phase) and second component (or liquid of the second phase) are immiscible.

There is also provided according to the invention a method of making a composition of the invention, which comprises mixing an oil, an enzyme that is able to convert a substrate to release hydrogen peroxide, and a substance that includes a substrate for the enzyme to form the composition.

Methods of the invention may also comprise mixing a non-aqueous solvent, such as a polar, organic solvent.

A method of the invention may employ a rheometer to form compositions of the invention. A rheometer may allow for control of shear rate and temperature.

The enzyme, and the substance comprising a substrate for the enzyme may be dissolved in a non-aqueous solvent to form a first mixture. The emulsifier may be added to the first phase, lipophilic phase or oil to form a second mixture. The first mixture may then be added dropwise to the second mixture to form an emulsion, whilst being mixed using e.g. a rheometer or mixer.

To form an emulsion, mixing may occur at a shear rate of between 1500 1/s to 2500 1/s, such as 2000 1/s. Mixing may occur at 30 and 50° C., e.g. about 37° C.

There is further provided according to the invention a method of making a composition of the invention, which comprises mixing the enzyme, the substance that includes a substrate for the enzyme, liquid of the second phase, liquid of the first phase, and optionally an emulsifier, under a high rate of shear for sufficient time to form an emulsion.

More stable emulsions may be formed if the ingredients of the emulsion are pre-mixed before contact with the emulsifier. Thus, in some embodiments, the enzyme, the substance that includes a substrate for the enzyme, liquid of the second phase, and liquid of the first phase are pre-mixed under a high rate of shear before contacting the pre-mixed ingredients with the emulsifier and mixing of the mixture comprising the pre-mixed ingredients and the emulsifier under the high rate of shear.

In some embodiments, the enzyme and the substance that includes a substrate for the enzyme are dissolved in the liquid of the second phase to form a solution before contacting the solution with the liquid of the first phase.

The high rate of shear may be from 1000 1/s to 4000 1/s. We have found that emulsions made using methods of the invention are more stable when formed using a higher rate of shear, for example from >2000 1/s to 4000 1/s, >2000 1/s to 3500 1/s, >2500 1/s to 4000 1/s, or >2500 1/s to 3500 1/s.

Mixing of the enzyme, the substance that includes a substrate for the enzyme, liquid of the second phase, liquid of the first phase, and the emulsifier (if present) may be carried out at a temperature of 20° C. to 40° C., for example from 35° C. to 40° C. More stable emulsions may be formed when mixing of the enzyme, the substance that includes a substrate for the enzyme, liquid of the second phase, liquid of the first phase, and the emulsifier (if present) is carried out at higher temperatures, for example from >37.5° C. to 40° C., or 38° C. to 40° C.

In some embodiments of methods of the invention, the enzyme, the substance that includes a substrate for the enzyme, liquid of the second phase, liquid of the first phase, and the emulsifier (if present), are mixed under a high rate of shear for at least 5 minutes, for example for 5 to 30 minutes.

Methods of the invention may be used to form creams, for example by inclusion of a viscosity-increasing agent, such as a thickener or gelling agent, for example a hydrocolloid.

In some embodiments, a method of the invention further comprises mixing a viscosity-increasing agent with the enzyme, the substance that includes a substrate for the enzyme, liquid of the second phase, liquid of the first phase, and the emulsifier (if present), under the high shear rate to form a cream.

In some embodiments, the viscosity-increasing agent is, or comprises a hydrocolloid, for example a polysaccharide.

In some embodiments, the hydrocolloid is, or comprises a hydrocolloid thickener, such as starch, modified starch, xanthan, a galactomannan (such as guar gum, locust bean gum, and tara gum), gum Arabic or acacia gum, gum karaya, gum tragacanth, konjac maanan, or a cellulose derivative, such as carboxymethyl cellulose, methyl cellulose, or hydroxypropylmethyl cellulose.

In some embodiments, the hydrocolloid is, or comprises a hydrocolloid gelling agent, such as alginate, pectin, carrageenan, gelatin, gellan, agar, agarose, modified starch, or a cellulose derivative, such as methyl cellulose or hydroxypropylmethyl cellulose.

The hydrocolloid gelling agent may capable of forming a gel by ionotropic gelation in the presence of cations. In such embodiments, a method of the invention further comprises mixing the cations with the hydrocolloid gelling agent, the enzyme, the substance that includes a substrate for the enzyme, liquid of the second phase, liquid of the first phase, and the emulsifier (if present), under the high shear rate to form the cream.

In some embodiments, the hydrocolloid gelling agent capable of forming a gel by ionotropic gelation, the enzyme, the substance that includes a substrate for the enzyme, liquid of the second phase, liquid of the first phase, and the emulsifier (if present), are mixed to form a mixture prior to contacting the cations with the mixture.

In some embodiments the hydrocolloid gelling agent capable of forming a gel by ionotropic gelation is contacted with the liquid of the second phase, the enzyme, and the substance that includes a substrate for the enzyme, prior to contact with the liquid of the first phase.

In some embodiments the cations are, or comprise calcium ions.

In particular embodiments the hydrocolloid gelling agent capable of forming a gel by ionotropic gelation in the presence of cations is, or comprises alginate, carrageenan or pectin, for example alginate.

In a particular embodiment, the hydrocolloid gelling agent is provided in aqueous solution, and the second phase is glycerol, wherein the glycerol is present in sufficient amount to bind free water in the composition and thereby prevent the enzyme catalysing release of hydrogen peroxide from the substance that includes a substrate for the enzyme.

In some embodiments the hydrocolloid gelling agent that is able to form a gel by ionotropic gelation (for example, alginate) is pre-mixed with the substance that includes a substrate for the enzyme, liquid of the second phase, and liquid of the first phase under a high rate of shear before the pre-mixed ingredients are contacted with the emulsifier (if present) and the cations (for example, calcium ions), and the mixture comprising the pre-mixed ingredients, the emulsifier (if present), and the cations is mixed under the high rate of shear.

The emulsifier (if present) may be contacted with the pre-mixed ingredients before the cations (for example, calcium ions). The cations (for example, calcium ions) may be added dropwise.

The cations (for example, calcium ions) may be provided in aqueous solution. Alternatively, the cations may be provided in non-aqueous solution, using a non-aqueous solvent, such as ethanol or acetic acid.

In particular embodiments, calcium chloride is provided in ethanol, and sodium alginate is provided in aqueous solution, and the second phase is glycerol, and the glycerol is present in sufficient amount to bind the free water in the alginate solution and thereby prevent the enzyme catalysing release of hydrogen peroxide from the substance that includes a substrate for the enzyme. This prevents premature release of hydrogen peroxide until the composition is contacted with water, thereby providing a stable composition.

As above, a method of the invention may employ a rheometer to form a composition of the invention. A rheometer may allow for control of shear rate and temperature. Alternatively, a high rate of shear may be provided by use of an ultrasonic probe, or a homogeniser.

There is also provided according to the invention, a pharmaceutical composition comprising a composition of the invention and a pharmaceutically acceptable carrier, excipient or diluent.

There is also provided according to the invention a composition of the invention for use as a medicament.

There is further provided according to the invention a composition of the invention for use in the prevention or treatment of a microbial infection.

The prevention or treatment is preferably by topical administration of the composition. The prevention or treatment may be by administration to a subject's respiratory tract. The prevention or treatment may be by administration to the body cavity. The prevention or treatment may be by internal administration to a subject.

There is also provided according to the invention a method of prevention or treatment of a microbial infection, which comprises administering an effective amount of a composition of the invention to a subject in need of such treatment.

The invention also provides use of a composition of the invention in the manufacture of a medicament for the prevention or treatment of a microbial infection.

The microbial infection may be a viral infection, for example, a herpes simplex virus (HSV) infection. In other embodiments, the microbial infection may be a fungal infection, or a bacterial infection.

The microbial infection may be: a nasal infection, such as sinusitis or rhinosinusitis; a respiratory tract infection, such as an upper respiratory tract infection (e.g. tonsillitis, laryngitis or sinusitis) or a lower respiratory tract infection (e.g. bronchitis, pneumonia, bronchiolitis or tuberculosis); an infection associated with chronic obstructive pulmonary disease (COPD), cystic fibrosis, bronchiectasis, asthma, or an HIV/AIDS-associated respiratory infection, or a respiratory infection associated with terminal disease.

A composition of the invention may be used to prevent or treat a microbial infection that comprises a biofilm, or a microbe that is capable of forming a biofilm. The biofilm may comprise a biofilm-forming bacterium, fungus, or virus. The microbe that is capable of forming a biofilm may be a bacterium, fungus, or virus.

A composition of the invention may be used in an antimicrobial wipe, in disinfection, for example in hospital disinfection, or as an antimicrobial spray, for example as a topical prophylactic antimicrobial spray to disinfect part of the body of a patient prior to surgery.

A composition of the invention may include an antimicrobial agent. For example, hydrogen peroxide may be present if the composition is formed by contacting the enzyme with the substance in aqueous solution under conditions for conversion of the substrate by the enzyme, and then drying the composition to reduce its water content to a level where there is insufficient free water to allow the enzyme to convert the substrate. Preferably, however, the composition does not include any detectable hydrogen peroxide. Such composition may be formed, for example, by contacting the enzyme with the substrate in the absence of sufficient free water to allow the enzyme to convert the substrate. Examples of other antimicrobial agents that may be present in a storage-stable composition of the invention include: an antibiotic, an antiviral agent, or an anti-fungal agent.

The composition may be a medical grade or medical device grade composition, or a pharmaceutical grade composition.

Each component of the composition may be a natural substance (i.e. each component is derived or purified from a natural source). Compositions for use according to the invention which contain only natural ingredients provide an attractive alternative to drug-based antimicrobial formulations.

Advantageously the substance is a honey. The honey may be a medical grade or medical device grade honey. In some embodiments, the honey is a honey that has been treated to remove or inactivate catalase activity originally present in the honey. According to an embodiment of the invention, the substance is a pasteurised honey, and the enzyme is a glucose oxidase. According to some embodiments, the substance is a medical grade or medical device grade honey, and the enzyme is a medical grade or medical device grade enzyme, suitably glucose oxidase.

Honey is a natural product made by honey bees using nectar from flowers. It is a saturated or super-saturated solution of sugars. Honey is defined in the Codex Alimentarius international food standard as “the natural sweet substance produced by honey bees from the nectar of plants or from secretions of living parts of plants or excretions of plant sucking insects on the living parts of plants, which the bees collect, transform by combining with specific substances of their own, deposit, dehydrate, store and leave in the honey comb to ripen and mature” (Revised Codex Standard for Honey, 2001).

Nectar typically includes approximately 14% simple sugars (w/w), 1% phenol compounds, and 85% water. The phenol compounds give the honey its taste, aroma and colour. In the warm conditions of the hive, typically 36° C., the nectar would very quickly ferment. To prevent this, the nectar is mixed with secretions, containing enzymes, from the salivary and hypopharyngeal glands of foraging bees. In the hive the nectar is passed from bee to bee and more secretions are added before it is stored in the cells of the hive. The amount of enzymes present varies with the age, diet and physiological stage of the bees (when a bee is a forager its glands produce more digestive enzymes), strength of the colony, temperature of the hive, and the nectar flow and its sugar content.

The enzymes added to nectar by bees include diastase, which catalyses the conversion of starch to dextrin and sugar, Invertase, which catalyses the conversion of sucrose to fructose and glucose, and glucose oxidase, which catalyses the conversion of glucose to hydrogen peroxide and gluconic acid. Low doses of hydrogen peroxide prevent the growth of yeasts that would quickly ferment the nectar. As the bees progressively dry the nectar to form honey, the gluconic acid makes the honey acidic (between pH 3.5 and 4.5). Water is effectively trapped to the sugar molecules in the honey and is not available for further chemical reactions. The amount of ‘free’ water in honey is measured as the water activity (a_{w}). The range of a_{w} found in honey has been reported to be 0.47-0.70, with mean values of 0.562 and 0.589 (RCIEGG, M; BLANC, B, 1981, The water activity of honey and related sugar solutions. Lebensmittel-Wissenschaft and Technologie 14: 1-6). The a_{w} of ripened honey is too low to support the growth of any species, with no fermentation occurring if the water content is below 17.1% (Molan, P. C. (1992). The antibacterial activity of honey: 1. The nature of the antibacterial activity. Bee World, 73(1), 5-28). The acidity of the honey and the lack of free water prevent the further risk of fermentation, and stop the glucose oxidase working. Honey also contains variable amounts of catalase originating from the nectar.

A typical chemical composition of blossom honey is:

TABLE 1
Typical honey composition

Blossom honey

| | Average | Min-Max |
|---|---|---|
| Component | (% w/w) | (% w/w) |
| Water content | 17.2 | 15-20 |
| Fructose | 38.2 | 30-45 |
| Glucose | 31.3 | 24-40 |
| Sucrose | 0.7 | 0.1-4.8 |
| Other | 5 | |
| disaccharides | | |
| Total sugars | 79.7 | |
| Minerals | 0.2 | 0.1-0.5 |
| Amino acids, | 0.3 | 0.2-0.8 |
| Proteins | | |
| Acids | 0.5 | 0.2-0.8 |
| pH | 3.9 | 3.5-4.5 |

In addition, trace amounts of pollen are present, which can be used to identify the botanical origin of the honey, as well as the enzymes invertase, diastase, catalase, and glucose oxidase. There is also phytochemical component. This varies but is typically up to ˜1%, depending on the source of the honey.

Once diluted, the glucose oxidase present in natural honey is able to convert glucose substrate in the diluted honey to release hydrogen peroxide. However, the variability in the content of honey (particularly in the content of glucose oxidase activity, glucose, and catalase activity) means that honeys from different sources, or different harvests of honey from the same source, can be very variable in their antimicrobial effectiveness.

According to an embodiment of the invention, the honey may be pasteurised. Pasteurisation of honey inactivates the catalase and glucose oxidase activity present in the honey. Optionally, the pasteurised honey may be filtered to remove any particles (such as wax particles and bee wings) that may be in the honey post-harvest. To form a storage-stable composition of the invention, a glucose oxidase is contacted with the pasteurised honey once it has cooled to a temperature (suitably 35-40° C.) that will not inactivate the added glucose oxidase and at which the honey remains sufficiently liquid to facilitate mixing with glucose oxidase.

Honey can be pasteurised at a temperature that is sufficient for the heat inactivation of catalase activity. A suitable minimum temperature is from 60° C. to 80° C. This temperature should be maintained preferably for at least two minutes.

The control of the heat process may be important, since a bi-product of heating honey is the formation of HMF (HydroxyMethylFurfuraldehyde) which is used as an indicator of heat and storage changes in honey. HMF is formed by the breakdown of fructose in the presence of acid. Heat increases the speed of this reaction. The increase in speed is exponential with increasing heat. For every degree that the honey is raised above 40° C., close to the normal hive ambient temperature, HMF increases rapidly. HMF is not a harmful product. Jams, Molasses, Golden Syrup etc. can have levels of HMF 10 to 100 times that of honey. However HMF levels are used as an indication of degradation of honey and under the Codex Alimentarius Standard 40 mg/I is the maximum permissible level in the EU for table honey.

To prevent the build up of HMF it is preferred that the honey is raised rapidly to temperature levels to inactivate the catalase and then the honey is brought quickly down in temperature to a maximum of between 40 and 45° C. using a heat exchange mechanism.

No water is added during the process of this preferred embodiment, and so the resulting composition does not include sufficient free water to allow the glucose oxidase to convert the glucose present to release hydrogen peroxide. The storage-stable composition comprises: pasteurised honey, and added glucose oxidase. There is no detectable hydrogen peroxide present. The composition can be stored at ambient temperature for at least several days.

In other embodiments of the invention, the honey may be unpasteurised.

According to some preferred embodiments, the honey (pasteurised or unpasteurised) is a creamed honey. Creamed honey is a honey that has been processed to control crystallization. Creamed honey contains a large number of small crystals, which prevent the formation of larger crystals that can occur in unprocessed honey. A method for producing creamed honey was described in U.S. Pat. No. 1,987,893. In this process, raw honey is first pasteurised, then previously processed creamed honey is added to the pasteurized honey to produce a mixture of 10% creamed honey and 90% pasteurised honey. The mixture is then allowed to rest at a controlled temperature of 14° C. This method produces a batch of creamed honey in about one week. A seed batch can be made by allowing normal honey to crystallize and crushing the crystals to the desired size. Large scale producers have modified this process by using paddles to stir the honey mixture while holding the mixture at 14° C. In alternative creaming methods, the pasteurisation step may be omitted, with the honey instead being slowly warmed to 37° C.

In other embodiments of the invention, the honey (pasteurised or unpasteurised) is an uncreamed honey. For example, the honey may be a pasteurised, uncreamed honey.

The glucose oxidase may be a purified natural glucose oxidase preparation which is of medical grade or medical device grade for medical applications. The activity of the glucose oxidase may be selected depending on the desired rate of production of hydrogen peroxide following dilution of the composition. Several glucose oxidase preparations are commercially available (glucose oxidase is identified by the reference CAS:9001-37-0). Common microbial sources for glucose oxidase from non genetically modified organisms include selected strains of Aspergillus niger, Penicillium amagasakiense, Penicillium variabile, Penicillium notatum. Medical device grade glucose oxidase, from GMO Aspergillus niger, is available from Biozyme UK, activity 240iu/mg. Food standard glucose oxidase, from Aspergillus niger, is available from BIO-CAT INC, activity 15,000 Units/g. Non-Genetically Modified glucose oxidase is available from BIO-CAT INC, activity 12,000/g. Glucose oxidase (GO3B2), from Aspergillus niger, is available from BBI Enzymes Limited, activity 360 Units/mg. Contaminants: alpha amylase no greater than 0.05%, Saccharase no greater than 0.05%, maltase no greater than 0.05% and GO/Cat no less than 2000.

The enzyme activity (for example, the glucose oxidase activity) may range, for example, from 1-400 IU/mg, or 1-300 IU/mg, for example 250-280 IU/mg. The amount of enzyme used is likely to depend on several factors, including the desired use of the composition, the amount of any catalase activity present in the substance, the amount of substrate present in the substance, the desired level of hydrogen peroxide release, and the desired length of time for hydrogen peroxide release. A suitable amount of enzyme can readily be determined by a person of ordinary skill in the art, if necessary using a well diffusion assay, to determine the extent of hydrogen peroxide release for different amounts of enzyme. Suitable amounts of enzyme (such as glucose oxidase) may be from 0.0001% to 0.5% w/w of the composition. The amount of enzyme used may be selected so as to produce a composition for generating antimicrobial activity that is equivalent to a selected phenol standard (for example a 10%, 20%, or 30% phenol standard).

Compositions of the invention, particularly compositions in which the substance is honey (for example, unpasteurised honey), and the enzyme is glucose oxidase that is able to convert D-glucose in the honey to release hydrogen peroxide, may comprise at least 1 unit, and preferably up to 1500 units, of glucose oxidase per gram of the composition. The glucose oxidase is additional (i.e. added as a result of human intervention) to any glucose oxidase activity that may naturally be present in the substance.

A “unit” is defined herein as the amount of enzyme causing the oxidation of 1 micromole of glucose (or other enzyme substrate) per minute at 25 degrees centigrade at pH 7.0.

The Applicant has found that the antimicrobial potency of compositions of the invention may be increased simply by increasing the amount of glucose oxidase activity present in the composition.

In some embodiments of the invention, a composition of the invention comprises more than 15 units, for example at least 30 units, at least 50 units, or at least 100 units, and suitably less than 685 units, for example 100-500 units, of glucose oxidase per gram of the composition. Such compositions have been found to have superior antimicrobial properties than compositions with up to 15 units of glucose oxidase per gram of the composition. In particular, such compositions have increased potency against a wide range of microorganisms, including MSSA, MRSA, Group A and B Streptococci, Enterococcus, E. coli, E. coli ESBL, Serr. liquefaciens Amp C, Kleb. pneumoniae, Pseudomonas aeruginosa, Acinetobacter baumannii, and Candida albicans.

In other embodiments of the invention, a composition of the invention comprises at least 500 units, for example 500-1000 units, or 685-1000 units, of glucose oxidase per gram of the composition. Such compositions have been found to have even more superior antimicrobial properties. In particular such compositions have further increased potency against a wide range of microorganisms, including Staphylococcus aureus, MSSA, MRSA, Group A and B Streptococci, Enterococcus, E. coli, E. coli ESBL, Serr. liquefaciens Amp C, Kleb. pneumoniae, Pseudomonas aeruginosa, Acinetobacter baumannii, and Candida albicans.

Compositions of the invention can be used to treat any microbial infection that can be treated by hydrogen peroxide. Examples include infection caused by gram positive bacteria, gram negative bacteria, acid-fast bacteria, viruses, yeasts, parasitic or pathogenic micro-organisms or fungi. In particular, infections caused by the following micro-organisms may be treated: Escherichia coli, Staphylococcus aureus, Pseudomonas aeruginosa, Candida albicans, Propionibacterium acnes, Staphylococcus aureus, Staphylococcus epidermidis, Staphylococcus saprophytics, Beta haemolytic Streptococci Group A or B, Campylobacter coli, Campylobacter jejuni, Methicillin Resistant Staphylococcus Aureus (MRSA), Methicillin Sensitive Staphylococcus Aureus (MSSA), Botrytis cinerea, Mycobacterium tuberculosis, Cryptosporidium, Plasmodium, and Toxoplasma.

The pasteurisation process inactivates any enzyme activity present in the honey, and so there is no variability in catalase and substrate conversion activity between pasteurised honeys from different sources, or between different harvests of honey from the same source. The amount of substrate conversion activity can be controlled by addition of a purified glucose oxidase preparation with a defined amount and activity of the enzyme. Thus, the inherent variability in antimicrobial properties between different types and harvests of honey is considerably reduced, and the antimicrobial properties of honeys with low antimicrobial potency are improved.

Compositions of the invention may be administered at an appropriate frequency determined by the subject or a healthcare provider. Suitably compositions of the invention may be administered at least every several days, for example every week, but preferably several times a day, every day, or every other day.

The amount of a composition of the invention administered will depend on many factors, such as the strength of the antimicrobial properties of the composition, and on the age and condition of the subject to be treated. However, for many applications it is expected that each administration comprises 0.1-100 g, 0.5-100 g, 1-100 g, 2-100 g, 5-100 g, 0.1-10 g, 0.5-10, or 1-10 g of a composition of the invention.

According to preferred embodiments of the invention, a composition of the invention is sterile.

Compositions of the invention may be sterilised by any suitable means. The Applicant has found that compositions comprising glucose oxidase retain glucose oxidase activity (and, therefore, the ability to release hydrogen peroxide on dilution) following sterilisation by exposure to gamma irradiation. A suitable level of gamma irradiation is 10-70 kGy, preferably 25-70 kGy, more preferably 35-70 kGy.

Compositions of the invention preferably have not been sterilized by ozonation, and do not include ozone, or any components that have been subjected to sterilisation by ozonation. In particular, compositions of the invention should not comprise ozonized honey or ozonated oil.

Sterilised compositions of the invention that are stored away from exposure to light are expected to retain stability for at least six months. For example, such compositions may be packaged in high-density polyethylene/low-density polyethylene (HDPE/LDPE) tubes or in polyester-aluminium-polyethylene (PET/AI/PE) sachets.

A composition of the invention is preferably a medical grade or medical device grade composition. Preferably the unrefined natural substance is a honey, suitably a medical grade or medical device grade honey.

Preferably a composition of the invention comprises a creamed honey, more preferably a creamed unpasteurised honey. Such compositions can readily be administered topically because the presence or number of large crystals has been minimised by the creaming process.

For compositions of the invention that comprise honey, it will be appreciated that there may be no need to use pasteurised honey in the composition if the composition is sterilised. It may instead be preferable to use unpasteurised honey (preferably creamed honey) or other unrefined natural substance. In some embodiments, compositions of the invention comprise unpasteurised honey, and added purified glucose oxidase.

Thus, a storage-stable composition of the invention may comprise unpasteurized honey, and added purified glucose oxidase that, in the presence of sufficient free water, is able to convert D-glucose in the honey to release hydrogen peroxide, wherein the composition does not include sufficient free water to allow the glucose oxidase to convert the D-glucose.

Such compositions may comprise at least 1 unit, and for example up to 1500 units, of glucose oxidase per gram of the composition. Suitably such compositions comprise more than 15 units of glucose oxidase per gram of the composition, for example at least 100 units, or 100-500 units, of glucose oxidase per gram of the composition, or at least 500 units, or 500-1000 units, of glucose oxidase per gram of the composition.

The honey of such compositions may comprise a creamed unpasteurized honey.

A composition of the invention may be a pharmaceutical composition comprising a pharmaceutically acceptable carrier, excipient, or diluent.

A composition of the invention may be in a form suitable for administration to a human or animal subject. Suitable forms include forms adapted for topical administration. Forms suitable for topical administration include a topical ointment, cream, lotion, oil, liniment, liquid, gel, or a dissolvable strip. If a storage-stable composition is used, this may be diluted by liquid present at the site of administration (for example, by saliva), leading to release of hydrogen peroxide at the administration site.

A composition of the invention may be present with at least one suitable antimicrobial or immunostimulatory component, excipient or adjuvant, or any other suitable component where it is desired to provide ability to generate antimicrobial activity. Preferably, however, the compositions do not include any antibiotic.

A composition of the invention may comprise “Surgihoney”. Surgihoney is unpasteurised honey with added purified glucose oxidase. Three different preparations of Surgihoney have been made with different antimicrobial potencies:

SH1 Surgihoney: unpasteurised honey with 0.1% (w/w) added glucose oxidase. The enzyme used was food grade glucose oxidase, from Aspergillus niger, from BIO-CAT, INC, activity 15,000 Units/g. Sealed sachets of the SH1 Surgihoney were gamma irradiated at a target dose of 11/6-14.2 kGy.

SH2 Surgihoney: unpasteurised honey with 0.1% (w/w) added glucose oxidase. The enzyme used was glucose oxidase (GO3B2), from Aspergillus niger, from BBI Enzymes Limited, activity 274 Units/mg. Unit Definition: the amount of enzyme causing the oxidation of 1 micromole of glucose per minute at 25 degrees centigrade at pH 7.0. Contaminants: alpha amylase no greater than 0.05%, Saccharase no greater than 0.05%, maltase no greater than 0.05% and GO/Cat no less than 2000.

SH3 Surgihoney: unpasteurised honey with 0.25% (w/w) added glucose oxidase. The enzyme used was glucose oxidase (GO3B2) from BBI Enzymes Limited, activity 274 Units/mg.

Thus, SH1 Surgihoney contains 15 units of glucose oxidase per gram of the composition, SH2 Surgihoney contains 274 units of glucose oxidase per gram of the composition, and SH3 Surgihoney contains 685 units of glucose oxidase per gram of the composition.

In some embodiments, a composition of the invention (in particular, a composition of the invention that comprises honey and added glucose oxidase—referred to below as “Active honey”) is not, or does not comprise the following:

Pommade

Active honey 25%

White petrolatum

Light liquid paraffin

Talc

Kaolin

Zinc oxide

In some embodiments, a composition of the invention (in particular, a composition of the invention that comprises honey and added glucose oxidase—referred to below as “Active honey”) is not, or does not comprise the following:

Lip Balm

| | |
|---|---|
| Petrolatum 5594 | 50% |
| Microcrystalline Wax | 9% |
| Cyclomethicone D5 | 31% |
| Active Honey | 10% |

In some embodiments, a composition of the invention (in particular, a composition of the invention that comprises honey and added glucose oxidase—referred to below as “Active honey”) is not, or does not comprise the following:

Cream

| | |
|---|---|
| Honey | 15% |
| Carbomer | 2.63% |
| Dimethicone | 0.13% |
| Disodium Lauryl Sulphosuccinate | 0.05% |
| Disodium Edetate | 0.13% |
| Glycerol | 5.26% |
| Silica Colloidal Hydrated | 0.33% |
| Poloxamer | 0.26% |
| Sodium Hydroxide | 0.41% |
| Purified Water | 85.03% |

In some embodiments, a composition of the invention does not comprise:

A cream formulation comprising Surgihoney SH1 was made with the following ingredients:
beeswax (for the lipophilic phase);
soya lecithin (as an emulsifier);
water; and
SH1 Surgihoney.

In some embodiments, a composition of the invention does not comprise an emulsion comprising the following ingredients:
10 g Surgihoney dissolved in 10 ml of glycerol;
10 ml of paraffin oil;
1 ml Polyglycerol polyricinoleate (PGPR).

In some embodiments, a method of the invention does not comprise the following method for preparation of a Surgihoney emulsion, as recited in Example 7:

10 g Surgihoney was dissolved in 10 ml of glycerol. 10 ml of paraffin oil was then added to a Rheometer (TA Instruments AR-G2) which had a Jacket Peltier and vane geometry attached. 1 ml of PGPR (Polyglycerol polyricinoleate) was then added. The rheometer was then started under the following conditions; Shear rate 2000 1/s, Temperature set at 37.5° C. After 2 minutes, 10 ml of Surgihoney-glycerol solution was added dropwise. Once a total of 10 minutes had elapsed the emulsion was transferred from the Jacket Peltier to a container.

Embodiments of the invention are now described, by way of example only, with reference to the accompanying drawings in which:

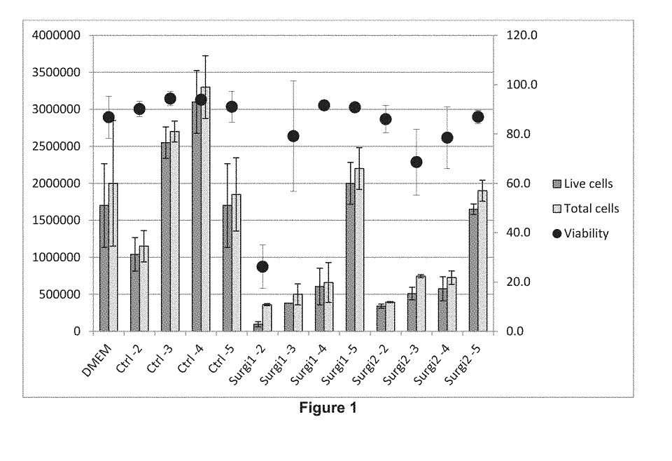

FIG. 1 shows the results of an assay for the cytotoxic activity of Surgihoney;

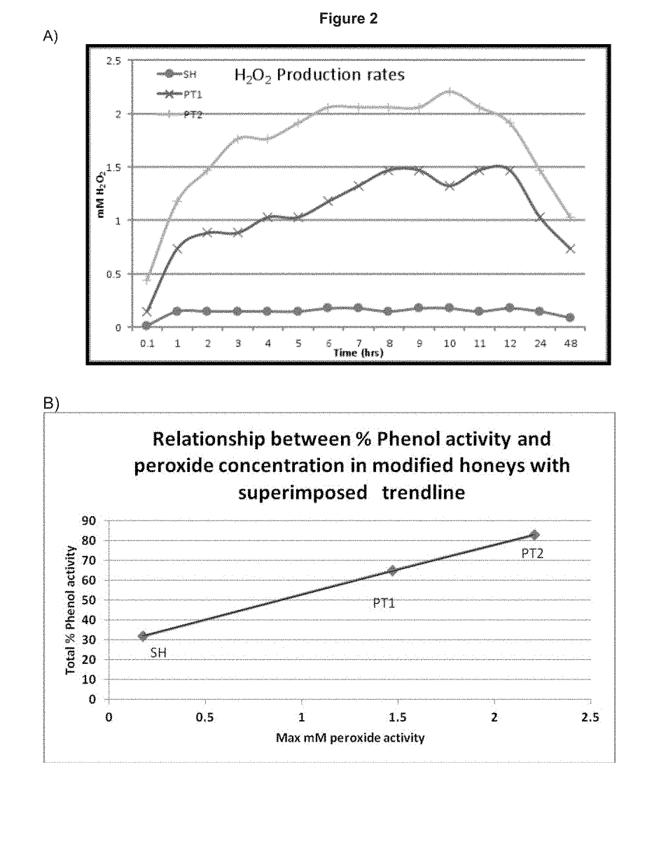

FIG. 2A shows different hydrogen peroxide production rates for Surgihoney SH1, SH2, and SH3;

FIG. 2B shows the relationship between phenol activity and maximum hydrogen peroxide activity in Surgihoney SH1, SH2, and SH3;

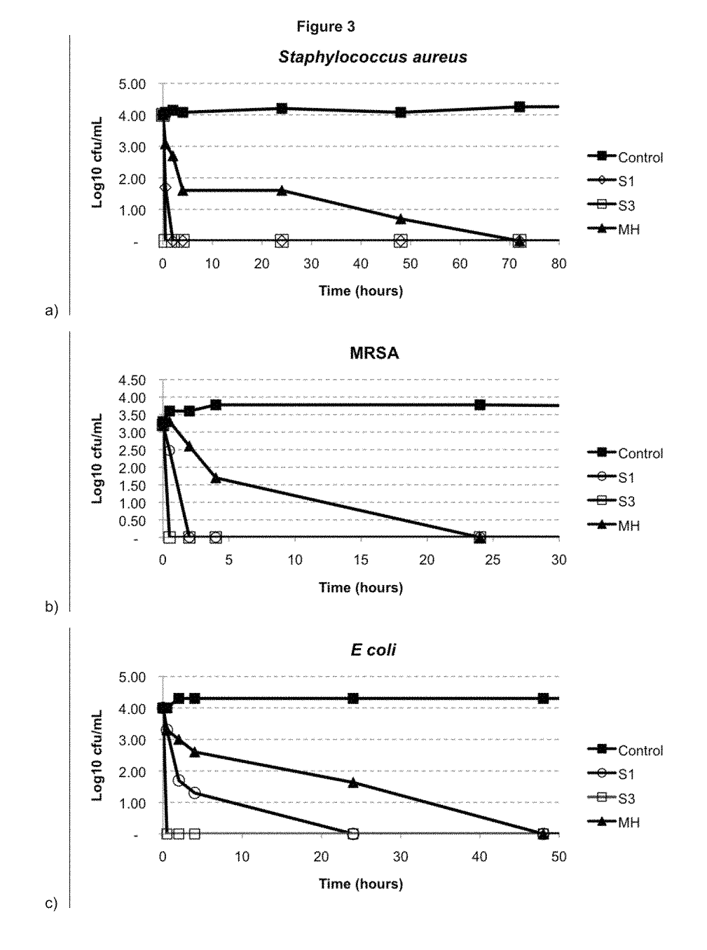

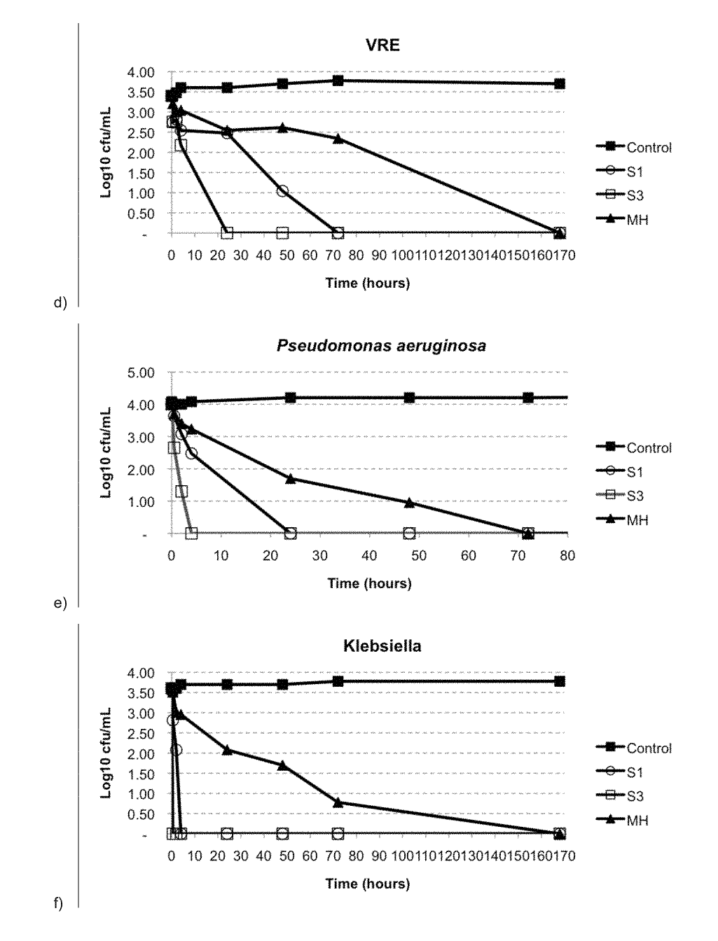

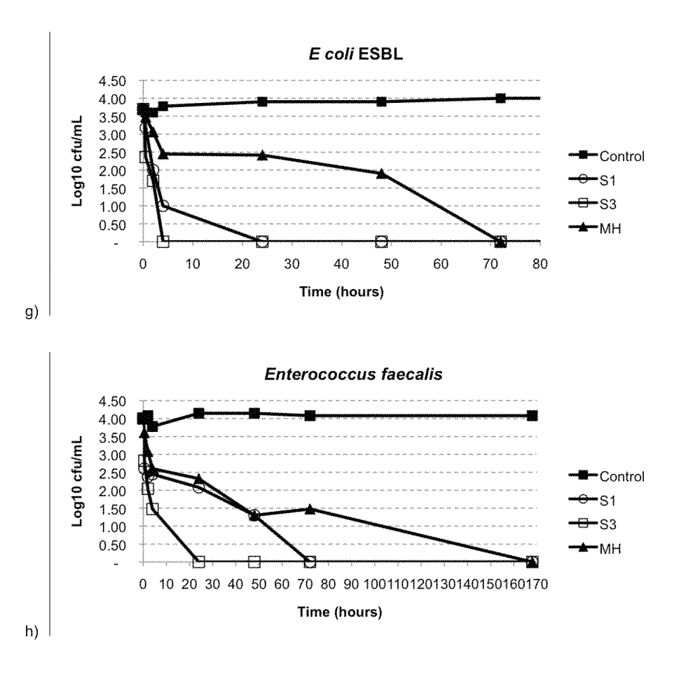

FIG. 3 shows time kill curves for Surgihoney 1 (S1), Surgihoney 3 (S3), and Medihoney (MH) for different test organisms: (a) Staphylococcus aureus; (b) Methicillin-resistant Staphylococcus aureus (MRSA); (c) E. coli; (d) vancomycin resistant enterococcus (VRE); (e) Pseudomonas aeruginosa; (f) Klebsiella; (g) E. coli ESBL; (h) Enterococcus faecalis; and

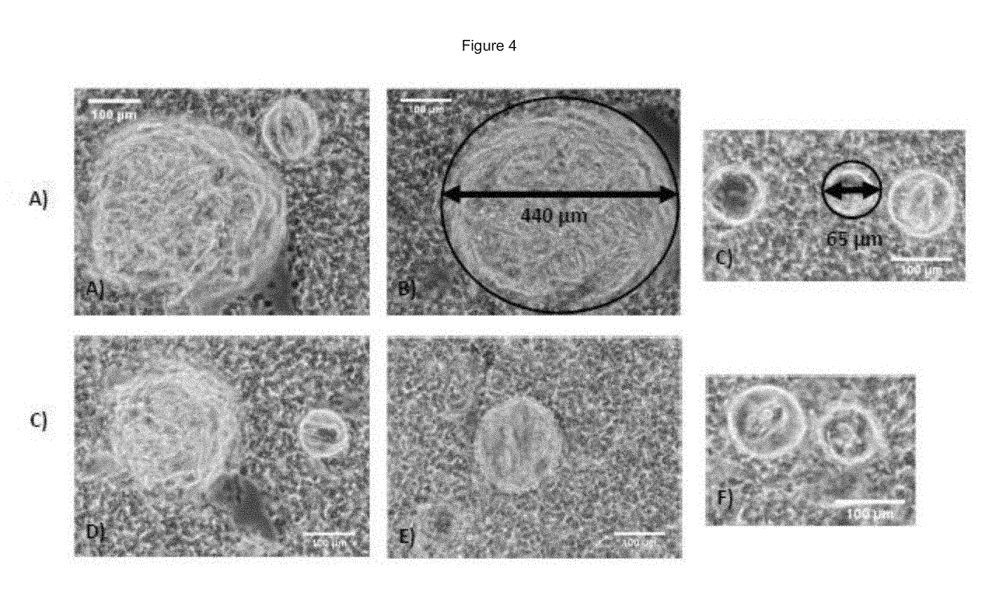

FIG. 4 shows an optical microscopy images of reverse micelles in an emulsion containing Surgihoney.

### EXAMPLE 1

Surgihoney Cream Formulation

A cream formulation comprising Surgihoney SH1 was made with the following ingredients:
beeswax (for the lipophilic phase);
soya lecithin (as an emulsifier);
water; and
SH1 Surgihoney.

The cream remains stable for many years, as judged by its ability to produce hydrogen peroxide when contacted with water.

### EXAMPLE 2

Anti-Viral Activity of Surgihoney

SH1 or SH2 Surgihoney was mixed with Herpes Simplex Virus Type 1 or 2 (HSV 1 or HSV 2) in cell culture medium (a 50% mixture of honey and virus in cell culture medium) and then incubated for 1 hour at 37° C. A dilution series was then made from the mixture, and the dilutions were plated onto Vero cells. SH1 Surgihoney reduced the titre of virus by 1 log. SH2 Surgihoney was virucidal for both HSV 1 and HSV 2 (>6 log drop in titre). The experiment was repeatable.

### EXAMPLE 3

Anti-Viral Activity of Surgihoney

SH1 or SH2 Surgihoney was mixed with Herpes Simplex Virus (HSV) (50 μg honey and 50 μl virus) and incubated for 1 hour at 37° C. A dilution series (10⁻², 10⁻³, 10⁻⁴, 10⁻⁵) was then made from the mixture, and the dilutions were used in a plaque reduction assay. Controls with no honey, or with control honey were also performed. The number of viral plaques formed for each dilution was recorded. The results are shown in the Table below.

TABLE 2
Anti-viral effect of Surgihoney

| | Dilu- | Experiment 1 | | | Experiment 2 | | |
|---|---|---|---|---|---|---|---|
| Honey | tion | well 1 | well 2 | well 3 | well 1 | well 2 | well 3 |
| SH1 | −2 | * | * | * | * | * | * |
| | −3 | 1 | 1 | 5 | 0 | 0 | 0 |
| | −4 | 0 | 1 | 1 | 0 | 0 | 0 |
| | −5 | 0 | 0 | 0 | 0 | 0 | 0 |
| SH2 | −2 | * | * | * | * | * | * |
| | −3 | 0 | 0 | 0 | 0 | 0 | 0 |
| | −4 | 0 | 0 | 0 | 0 | 0 | 0 |
| | −5 | 0 | 0 | 0 | 0 | 0 | 0 |
| Control | −2 | 100 | 95 | 88 | 108 | 128 | 106 |
| Honey | −3 | 13 | 15 | 11 | 14 | 12 | 15 |
| | −4 | 2 | 1 | 2 | 3 | 2 | 2 |
| | −5 | 0 | 0 | 0 | 0 | 0 | 1 |
| No | −2 | | | | 160 | 158 | 164 |
| Honey | −3 | | | | 28 | 22 | 18 |
| | −4 | | | | 6 | 4 | 1 |
| | −5 | | | | 1 | 0 | 1 |

The results show that SH1 and SH2 Surgihoney was strongly virucidal against HSV in both experiments.

### EXAMPLE 4

Cytotoxic Activity of Surgihoney

SH1 or SH2 Surgihoney (50 μg honey diluted 10⁻², 10⁻³, 10⁻⁴, 10⁻⁵) was incubated on cells for 2 days. The number of live cells, and the total number of cells was counted (percentage viability=live/total×100). The results are shown in the table below, and in FIG. 1.

The results show that SH1 Surgihoney was cytotoxic at the 10⁻² dilution, and cytostatic at the 10⁻³ and 10⁻⁴ dilutions, and that SH2 Surgihoney was cytostatic at the 10⁻², 10⁻³ and 10⁻⁴ dilutions. SH1 and SH2 Surgihoney were not cytotoxic or cytostatic at the 10⁻⁵ dilution.

It is concluded from the results in Examples 3 and 4 that Surgihoney can be administered at doses which are virucidal but not cytotoxic or cytostatic.

TABLE 3
Cytotoxic activity of Surgihoney

| | | Number of live cells | | | | Total number of cells | | | | Percentage viability | | | |
|---|---|---|---|---|---|---|---|---|---|---|---|---|---|
| Condi- | Dilu- | | | | standard | | | | standard | | | | standard |
| tion | tion | rep1 | rep2 | Ave | deviation | rep1 | rep2 | Ave | deviation | rep1 | rep2 | Ave | deviation |
| DMEM | — | 1300000 | 2100000 | 1700000 | 565685.4 | 1400000 | 2600000 | 2000000 | 848528.1 | 92.9 | 80.8 | 86.8 | 8.5 |
| Control | −2 | 1200000 | 880000 | 1040000 | 226274.2 | 1300000 | 1000000 | 1150000 | 212132 | 92.3 | 88.0 | 90.2 | 3.0 |
| honey | −3 | 2700000 | 2400000 | 2550000 | 212132 | 2800000 | 2600000 | 2700000 | 141421.4 | 96.4 | 92.3 | 94.4 | 2.9 |
| | −4 | 3400000 | 2800000 | 3100000 | 424264.1 | 3600000 | 3000000 | 3300000 | 424264.1 | 94.4 | 93.3 | 93.9 | 0.8 |
| | −5 | 2100000 | 1300000 | 1700000 | 565685.4 | 2200000 | 1500000 | 1850000 | 494974.7 | 95.5 | 86.7 | 91.1 | 6.2 |
| SH1 | −2 | 120000 | 70000 | 95000 | 35355.34 | 370000 | 350000 | 360000 | 14142.14 | 32.4 | 20.0 | 26.2 | 8.8 |
| | −3 | 380000 | 380000 | 380000 | 0 | 400000 | 600000 | 500000 | 141421.4 | 95.0 | 63.3 | 79.2 | 22.4 |
| | −4 | 430000 | 780000 | 605000 | 247487.4 | 470000 | 850000 | 660000 | 268700.6 | 91.5 | 91.8 | 91.6 | 0.2 |
| | −5 | 1800000 | 2200000 | 2000000 | 282842.7 | 2000000 | 2400000 | 2200000 | 282842.7 | 90.0 | 91.7 | 90.8 | 1.2 |
| SH2 | −2 | 320000 | 360000 | 340000 | 28284.27 | 390000 | 400000 | 395000 | 7071.068 | 82.1 | 90.0 | 86.0 | 5.6 |
| | −3 | 450000 | 570000 | 510000 | 84852.81 | 760000 | 730000 | 745000 | 21213.2 | 59.2 | 78.1 | 68.6 | 13.3 |
| | −4 | 460000 | 690000 | 575000 | 162634.6 | 660000 | 790000 | 725000 | 91923.88 | 69.7 | 87.3 | 78.5 | 12.5 |
| | −5 | 1600000 | 1700000 | 1650000 | 70710.68 | 1800000 | 2000000 | 1900000 | 141421.4 | 88.9 | 85.0 | 86.9 | 2.7 |

### EXAMPLE 5

Antimicrobial Activity of Surgihoney

The antimicrobial activity of Surgihoney (SH) and two prototype modified honeys made by Apis mellifera (honeybee) against Staphylococcus aureus (NCIMB 9518) was tested. We also examined a number of modified types of Surgihoney for the ability to change the level of production of hydrogen peroxide from the samples.

Methods: Surgihoney (SH) was compared with two modified honeys, Prototype 1 (PT1) and Prototype 2 (PT2) using a bioassay method against a standard strain of Staphylococcus aureus. Further work studied the rate of generation of hydrogen peroxide from these preparations.

Results: Surgihoney antimicrobial activity was shown to be largely due to hydrogen peroxide production. By modification of Surgihoney, two more potent honey prototypes were shown to generate between a two- and three-fold greater antibacterial activity and up to ten times greater peroxide activity.

Conclusions: Surgihoney shows good antimicrobial activity. Two further honey prototypes have been shown to have antimicrobial activity that is possible to be enhanced due to demonstrated increases in peroxide activity.

Methods

1. Determination of Honey Activity by Bioassay Method

The antibacterial activity of Surgihoney (S) and two modified honeys, Prototype 1 (PT1) and Prototype 2 (PT2) was measured using Staphylococcus aureus (NCIMB 9518) and expressed as the equivalent percentage phenol. Values were calculated of the mean from three sample replicates tested, repeated on three days.

Assay Method. The agar well diffusion method used was adapted from the punch plate assay for inhibitory substances described in the Microbiology Standard Methods Manual for the New Zealand Dairy Industry (1982) [Bee Products Standards Council: Proposed standard for measuring the non-peroxide activity of honey. In. New Zealand: Bee Products Standards Council; 1982.].

Inoculum Preparation. Overnight culture was adjusted to an absorbance of 0.5 measured at 540 nm using sterile nutrient broth as a blank and a diluents and a cuvette with a 1 cm pathway.

Assay Plate preparation. A volume of 100 μl of the culture adjusted to 0.5 absorbance was used to seed 150 ml nutrient agar to make the assay plates. The agar was swirled to mix thoroughly and poured into large petri dishes which had been placed on a level surface. As soon as the agar was set the plates were placed upside down overnight before using the next day. For assay these seeded plates were removed from 4° C. and allowed to stand at room temperature for 15 min before cutting 7.0 mm diameter wells into the surface of the agar. 250 μl of test material (sample or standard) was placed into each well.

Catalase solution. A 200 mg/ml solution of catalase from bovine liver (Sigma C9322, 2900 units/mg) in distilled water was prepared fresh each day.

Sample preparation. Primary sample solutions were prepared by adding 4 g of sample to 4 ml of distilled water in universals and placed at 37° C. for 30 minutes to aid mixing. To prepare secondary solutions, 2 ml of the primary sample solution was added to 2 ml of distilled water in universals and mixed for total activity testing and 2 ml of the primary sample solution was added to 2 ml of catalase solution and mixed for non-peroxide activity.

Preparation of phenol standards. Standards (w/v) 10%, 30%, 50% phenol were prepared by dissolving phenol in water. Phenol standards were brought to room temperature in the dark before use and were mixed thoroughly before addition to test wells. Each standard was placed in three wells to test in triplicate. Standards were kept at 4° C. with an expiry date of one month.

Sample and standard application. All samples and standards were tested in triplicate by adding 250 μl to each of 3 wells.

Plate incubation. After application of samples the plates were incubated for approximately 18 hours at 37° C. The diameter of inhibition zones, including the diameter of the well (7.0 mm), was recorded.

Calculation of antibacterial activity of samples. The mean diameter of the clear zone around each phenol standard was calculated and squared. A standard graph was plotted of % phenol against the square of the mean diameter of the clear zone. A best-fit straight line was obtained using linear regression and the equation of this line was used to calculate the activity of each diluted honey sample from the square of the mean measurement of the diameter of the clear zone. To allow for the dilution (assuming the density of the Surgihoney to be 1.35 g/ml) this figure was multiplied by a factor of 4.69 and the activity of the samples was then expressed as the equivalent phenol concentration (% w/v).

Total Activity: all the activity, including activity due to hydrogen peroxide (H₂O₂).

Non-Peroxide Activity: H₂O₂ is removed by treating samples with catalase enzyme.

2. Determination of Honey Activity by H₂O₂ Method

The activity was measured using the Merckoquant®, 1.10011. & 1.10081.

Peroxide Test Kits. Concentrations expressed as the equivalent mg/L H₂O₂.

Samples were diluted 1:10 with purified water. Following 5 min incubation, all samples were measured for H₂O₂ production each hour over a 12 hour period followed by 24 and 48 hour time points.

Method of Determination. Peroxidase transfers oxygen from the peroxide to an organic redox indicator, which is then converted to a blue coloured oxidation product. The peroxide concentration is measured semi-quantitatively by visual comparison of the reaction zone of the test strip with the fields of a colour scale. The reaction zone of the test strip is immersed into the Surgihoney sample for 1 sec, allowing excess liquid to run off the strip onto an absorbent paper towel and after 15 seconds (Cat. No. 110011), 5 seconds (Cat. No. 110081), after which a determination of the colour formed in the reaction zone more precisely coincided with the colour fields scale.

Results

1. Activity Rating

The antimicrobial activity produced by the modification of the honey samples resulted in a two-fold and almost three-fold respectively increase in phenol activity with PT1 and PT2 compared with Surgihoney alone. The results for the three samples of Surgihoney (SH) and two modified prototypes, PT1 and PT2 are shown in the Table below.

TABLE 4
The peroxide and non-peroxide antibacterial activities of
Surgihoney (SH) and two modified prototypes, PT1 and PT2
against Staphylococcus aureus (NCIMB 9518).

| | | Total | |
|---|---|---|---|
| | | Activity (% | Non-Peroxide |
| Sample Name | Batch No. | phenol) | Activity (% phenol) |
| Surgihoney | 2015-06-018B | 32 | 0 |
| Surgihoney PT1 | HHI4110311 | 65 | 7 |
| Surgihoney PT2 | HHI14110312 | 83 | 10 |

2. Determination of Honey Activity by H₂O₂ Method

The prototype modifications are observed to generate up to seven and ten times the hydrogen peroxide activity of Surgihoney. The results for the three samples are shown in FIG. 2A. By taking the maximum level of hydrogen peroxide output for each of the three honey prototypes and plotting this against the total phenol activity a linear relationship is observed (FIG. 2B).

Discussion

The results from this work show that the main antimicrobial activity of Surgihoney and two modified prototypes, PT1 and PT2 are due to hydrogen peroxide. This is a similar finding to certain other honeys from a variety of floral sources. However, unlike previous work the availability of hydrogen peroxide from the samples is able to be enhanced and at 12 hours is seven and ten times respectively the value for Surgihoney alone. There is a striking linear relationship between the antimicrobial activity and the maximum output of hydrogen peroxide from the three honey prototypes.

This peroxide activity offers potent antimicrobial activity that is ideally suited to treat or prevent microbial infections. Hydrogen peroxide is an effective antimicrobial and is already used as a biocide for its potent activity against vegetative bacteria, yeasts and spores. It produces its antimicrobial effect through chemical oxidation of cellular components.

The human toxicity of hydrogen peroxide is concentration dependent and one study has claimed that the differential concentrations for antimicrobial and human toxicity might overlap. By contrast, certain preparations of honey have been shown to be an effective antimicrobial agent by supplying low concentrations of hydrogen peroxide continuously over time rather than as a large amount and without such toxicity. Indeed there is compelling evidence that where physiological levels of hydrogen peroxide are applied to mammalian cells there is a stimulation of biological responses and activation of specific biochemical pathways in these cells.

Clearly Surgihoney and the two modified prototypes, PT1 and PT2 offer effective hydrogen peroxide release over at least 24 hours.

Conclusions

Surgihoney and the two modified prototypes, PT1 and PT2 have been shown to have potent antimicrobial activity against a standard strain of Staphylococcus aureus. These antimicrobial activities have been shown to be due to hydrogen peroxide. The activity is scalable and can be described in terms of hydrogen peroxide activity. These modified honeys are effective, non-toxic and easy to administer.

### EXAMPLE 6

In Vitro Antimicrobial Activity of Surgihoney

This example describes susceptibility testing of a range of bacterial isolates to Surgihoney by disc diffusion method, minimum inhibitory concentration (MIC) and minimum cidal concentration (MBC) determination, and time bactericidal measurements.

Summary

Results: Surgihoney demonstrates highly potent inhibitory and cidal activity against a wide range of Gram positive and Gram negative bacteria and fungi. MIC/MBC's are significantly lower than concentrations likely to be achieved in topical clinical use. Surgihoney 1 MIC/MBC's for Staph. Aureus are 31 and 125 gms/L and Surgihoney 3 MIC/MBC's 0.12 and 0.24 gms/L.

Cidal speed depends on the potency. In Surgihoney 1, the least potent, complete cidal activity occurs for all organisms tested within 48 hours. For Surgihoney 3, the most potent, cidal activity occurs within 30 minutes. Maintenance of the Surgihoney inoculums preparation for up to a week demonstrated complete cidal activity and no bacterial persistence.

Conclusions: Surgihoney has wide potential as a highly active topical treatment combining the effects of the healing properties of honey with the potent antimicrobial activity of the bioengineered product. It is highly active against multidrug resistant bacteria. It is more active than other honeys tested and comparable to chemical antiseptics in antimicrobial activity.

This study examines the in-vitro properties of Surgihoney. Surgihoney retains all the established healing properties of natural honey but its antimicrobial activity can be set at whichever potency is required. This study determined minimum inhibitory concentrations (MIC) and minimum bactericidal concentrations (MBC) of Surgihoney 1, 2 and 3 and time kill curves.

Methods

Surgihoney was provided as potency grades 1, 2 and 3. It was presented as a sterile pharmaceutical grade product in a sachet in semisolid form.

Clinical isolates were collected from soft tissue microbiology samples. Eighteen isolates of Staphylococcus aureus, 12 methicillin-sensitive (MSSA) and 6 methicillin-resistant (MRSA), 6 isolates of β haemolytic streptococci, Lancefield groups A (2), B (2), C (1), G (1), 5 isolates of Enterococcus spp. Including vancomycin-resistant E. faecium, 6 of Esch. coli, including extended spectrum β lactamase producers, 2 of Klebsiella spp., 1 Serratia Marcescens Amp C producer, 4 of Pseudomonas aeruginosa, 1 of Acinetobacter Iwoffii, 1 of Propionibacterium acnes, 1 Bacteroides fragilis, and 2 of Candida albicans, 1 of Candida glabrata, 1 of Aspergillus fumigates were tested against Surgihoney.

Agar Diffusion

Six mm wells were cut in isosenitest agar which had already been inoculated with the test organism at a concentration to give a semiconfluent growth. Test Surgihoney and other honeys in the pilot study were added to the wells.

A pilot study was carried out initially to compare Surgihoney potencies S1, S2, S3 with a variety of honeys from around the world, European, South American, New Zealand, Yemani, Sudanese and with medical honey, Medihoney and with antimicrobial dressings containing silver (Silver Aquacell) and iodine (Iodoflex). Wells were cut in the plates inoculated with Staphylococcus aureus and filled with test honey or in the case of the dressings, these were cut to 2×2 cm and placed on the surface of the inoculated plates.

Following the pilot studies the Surgihoney potencies S1, S2, S3 were tested alone against the range of bacterial isolates from skin lesions. The wells were filled to the surface with a preparation of approximately 2 gms neat Surgihoney of the three potencies, diluted and emulsified in an equal volume of sterile water. Zone sizes were measured after 18-24 hours aerobic incubation (longer for Candida and Aspergillus spp., and anaerobically for Propionibacterium sp. And Bacteroides sp.)

Minimum Inhibitory Concentrations and Minimum Bactericidal Concentrations

Surgihoney product was warmed to 37° C. to liquefy it and 5 gms was mixed with 10 mL sterile deionised water. This dilution was regarded as the ‘neat’ substance for serial dilution. The British Society of Antimicrobial Chemotherapy (BSAC) method for performing minimum inhibitory concentrations (MIC's) and minimum bactericidal concentrations (MBC's) was used (Andrews J M. Determination of minimum inhibitory concentrations. J Antimicrob 372 Chemother 2001; 48(Supp 1): 5-16). The Surgihoney products were serially diluted in microtitre tray wells from neat to 1 in 1024. 75 μL of each honey dilution was added to each well in the strip of the microtitre tray. The neat concentration represented a concentration of 250 gm/L and the 1 in 2048 dilution, approximately 0.12 gm/L.

The test organisms were prepared by taking four morphologically identical colonies for each organism from pure culture to create a 0.5 McFarland density. This was further diluted 1:10.

All wells including controls were inoculated with 75 μL of the test isolate preparation. The well trays were incubated at 37° C. for 18 hours. The MIC was regarded as the most dilute well that showed no detectable turbidity.

The MIC well and those around the MIC well were sub-cultured on blood agar and incubated at 37° C. for 18 hours to determine the MBC. The MBC was the most dilute concentration which showed no growth after incubation.

Time Kill Curves

The test organism inoculums was prepared by taking 0.1 mL of a 0.5 MacFarlane density of the test organism and inoculating this in 3 mL of nutrient broth. The test inoculums was divided into 3 separate bijous, a control and three test preparations to which were added 0.5 g of Surgihoney 1 (S1), Surgihoney 3 (S3) or Medihoney (MH). Colony counts of the inocula were determined by serial dilution 1:10 and plating 0.1 mL on a blood agar plate, repeated 3 times.

The test and control inocula were kept at 30° C. to simulate the temperature of a superficial skin lesion. Colony counts were performed as above in triplicate at time 0.5, 2, 4, 24, 48, 72 and 168 hours.

A terminal culture was performed by inoculating 0.1 ml of the original inoculums into nutrient broth to neutralise any residual effect of the Surgihoney and incubating for 72 hours at 37° C., before plating on blood agar to determine test organism survival.

Results

Inhibitory Zone Sizes.

The pilot comparative studies demonstrated that all the Surgihoney potencies had greater antimicrobial activity than any other honey tested including the medical grade honey, Medihoney. The inhibitory zones for S1 were larger than those produced by any other honey. Silver dressings produced some inhibitory effect beneath the dressing but there was no zone of inhibition as there was for Surgihoney. Iodine dressings produced a large zone of inhibition (approximately 70 mm) to Staphylococcus aureus, larger than S1 (36 mm) and equivalent to S3 (67 mm).

In the quantitative zone size testing, Surgihoney at all potencies produced an inhibitory zone in agar diffusion against all bacteria tested, both Gram positive and Gram negative bacteria including multiply antibiotic resistant bacteria, and fungal species. The zone size for each species increased with increasing Surgihoney potency preparations. Table 5. The inhibitory effect of Surgihoney was not dependant only on direct contact with the active agent as with the silver dressings, but diffused well beyond the well producing the extensive zones listed in Table 5.

MIC's & MBC's

Surgihoney demonstrated significant antimicrobial activity against all the isolates tested. MIC's and MBC's were very consistent amongst isolates of the same species whether the isolates were multidrug resistant or highly sensitive. Table 6 lists the MIC and MBC values for isolate species tested by dilution ratio and Table 7 shows the MIC and MBC's in grams per litre. The degree of potency rose with the grade of Surgihoney. The MBC for each isolate was close to the MIC within a single dilution in most cases.

Topical concentration of Surgihoney is estimated at approximately 500 gms/L. Surgihoney 1 MIC/MBC's for Staph. Aureus are 31 and 125 gms/L and Surgihoney 3 MIC/MBC's 0.12 and 0.24 gms/L respectively.

Time Kill Curves.

Surgihoney kills bacteria rapidly. Starting with a colony forming units per millilitre (cfu/mL) of approximately 105, cfu/mL numbers in the control rose steadily, whereas in the Surgihoney inocula the cfu/mL fell rapidly after contact with both potencies of Surgihoney. By 30 minutes cfu numbers had fallen 1000 fold in most cases for both S1 and S3 (FIG. 3). For S1 bacterial growth was undetectable by 2 hours in most cases and for S3 by 30 minutes. Enterococci appeared more resilient and persisted for 48 hours. Cidal activity was complete for all organisms as terminal culture in nutrient broth with subsequent plating on blood agar failed to detect any organism in the S1 or S3 inocula.

Discussion

Surgihoney is natural honey which is also organic in the current sense of the word in that it has no agricultural additives or antimicrobial residues unlike much commercial honey for human consumption. It is not dependant on particular nectar sources, unlike honeys such as manuka which depends on a specific plant nectar source for its enhanced activity. The antimicrobial activity can be controlled in Surgihoney by the preparation process allowing the production of different grades with measured potency which is consistent.

This study has clearly demonstrated the efficacy of Surgihoney as a highly potent antimicrobial, active against all species of bacteria and fungi tested. In the preliminary pilot studies comparing Surgihoney with a variety of honeys sourced from around the world and with medical grade honey, Medihoney, Surgihoney demonstrated significantly greater antimicrobial efficacy. By comparison with the commonly used topical antiseptics silver and iodine, Surgihoney 3 produced an antimicrobial effect as great as iodine dressings and greater than silver dressings (Aquacel Ag) which was only effective at inhibiting bacteria in direct contact with the dressing.

MIC and MBC testing show that Surgihoney not only inhibits but also kills microbes at concentrations 10 to a 1000 fold below those that are likely to be achieved in topical treatment, estimated at 500 gms/L. The cidal activity of Surgihoney occurs at concentrations close to its inhibitory activity. There is therefore the potential for Surgihoney to be highly active in polymicrobial inhibition and eradication when applied topically.

The speed of cidal activity is shown by the time kill curves to be extremely rapid, within 30 minutes for Surgihoney 3 and within 2 hours for Surgihoney 1. This is the case for both Gram-positive and Gram-negative organisms, although enterococci appear slightly more resilient. Fungi, Candida spp. Aspergillus sp. also require higher concentrations and more prolonged exposure to inhibit growth and kill the organism.

These in vitro studies have demonstrated the potential of Surgihoney with high antimicrobial activity whose potency can be controlled.

Conclusion

These in vitro results support the clinical use of Surgihoney as a potent and non-toxic antimicrobial.

TABLE 5
Inhibitory zones sizes with different potencies of Surgihoney (S1, S2, S3)

| | | S1 | S2 | S3 |
|---|---|---|---|---|
| | No. of | Mean zone | Mean zone | Mean zone |
| Bacteria | strains | (range)/mm | (range)/mm | (range)/mm |
| Methicillin-sensitive | 12 | 36.2 (32-38) | 53.4 (44-58) | 66.5 (60-72) |
| Staphylococcus | | | | |
| aureus (MSSA) | | | | |
| Methicillin-resistant | 6 | 35.6 (31-38) | 52.6 (48-59) | 67.3 (59-73) |
| Staphylococcus | | | | |
| aureus (MRSA) | | | | |
| Streptococci Beta | 6 | 40.0 (35-42) | 44.5 (38-51) | 59.2 (53-69) |
| haemolytic | | | | |
| Enterococcus spp | 5 | 38.0 (34-39) | 49.5 (44-55) | 61.8 (59-64) |
| Escherichia coli | 6 | 33.4 (30-37) | 49.5 (36-55) | 62.7 (59-69) |
| Klebsiella sp. | 2 | 34.2 (30-38) | 40.0 (38-42) | 57.0 (52-62) |
| Pseudomonas | 4 | 25.8 (20-28) | 34.8 (30-38) | 50.2 (46-51) |
| aeruginosa | | | | |
| Acinetobacter | 1 | 32.1 | 43.7 | 55.2 |
| lwoffii | | | | |
| Bacteroides fragilis | 1 | 22.3 | 28.7 | 34.2 |
| Propionibacterium | 1 | 19.7 | 23.4 | 31.9 |
| acnes | | | | |
| Candida sp. | 2 | 9 (8-10) | 15 (15) | 26 (24-28) |
| Aspergillus | 1 | 8 | 12 | 18 |
| fumigatus | | | | |

TABLE 6
Serial double dilutions from neat Surgihoney (S1, S2,
S3) showing dilution of minimum inhibitory concentration
(MIC) and minimum bactericidal concentration (MBC).

| | S1 | | S2 | | S3 | |
|---|---|---|---|---|---|---|
| Organism name | MIC | MBC | MIC | MBC | MIC | MBC |
| MSSA | 1:8 | 1:2 | 1:32 | 1:16 | 1:2048 | 1:1024 |
| MRSA | 1:16 | 1:4 | 1:32 | 1:16 | 1:2048 | 1:1024 |
| Group B | 1:64 | 1:16 | 1:64 | 1:64 | 1:1024 | 1:256 |
| Streptococci | | | | | | |
| Group A | 1:32 | 1:16 | 1:128 | 1:64 | 1:1024 | 1:512 |
| Streptococci | | | | | | |
| Enterococcus | 1:8 | 1:2 | 1:32 | 1:4 | 1:256 | 1:64 |
| E. coli | 1:8 | 1:4 | 1:64 | 1:64 | 1:256 | 1:128 |
| E. coli ESBL | 1:8 | 1:2 | 1:64 | 1:64 | 1:256 | 1:128 |
| Serr. | 1:8 | 1:4 | 1:16 | 1:4 | 1:256 | 1:128 |
| liquefaciens | | | | | | |
| Amp C | | | | | | |
| Kleb. | 1:4 | 1:2 | 1:32 | 1:32 | 1:256 | 1:128 |
| pneumoniae | | | | | | |
| Pseud. | 1:16 | 1:16 | 1:64 | 1:16 | 1:256 | 1:64 |
| aeruginosa | | | | | | |
| Candida | Turbid | Growth | 1:16 | 1:16 | 1:64 | 1:64 |
| albicans | at neat | at neat | | | | |

TABLE 7
Surgihoney MIC and MBC values expressed in Grams/Litre

| | S1 | | S2 | | S3 | |
|---|---|---|---|---|---|---|
| Organism name | MIC | MBC | MIC | MBC | MIC | MBC |
| MSSA | 31 | 125 | 7.8 | 15.6 | 0.12 | 0.24 |
| MRSA | 15.6 | 62.5 | 7.8 | 15.6 | 0.12 | 0.24 |
| Group B | 3.9 | 15.6 | 3.9 | 3.9 | 0.24 | 0.9 |
| Streptococci | | | | | | |
| Group A | 7.8 | 15.6 | 1.9 | 3.9 | 0.24 | 0.48 |
| Streptococci | | | | | | |
| Enterococcus | 31 | 125 | 7.8 | 62.5 | 0.9 | 3.9 |
| E. coli | 31 | 62.5 | 3.9 | 3.9 | 0.9 | 1.9 |
| E. coli ESBL | 31 | 125 | 3.9 | 3.9 | 0.9 | 1.9 |
| Serr. | 31 | 62.5 | 15.6 | 62.5 | 0.9 | 1.9 |
| liquefaciens | | | | | | |
| Amp C | | | | | | |
| Kleb. | 1:4 | 125 | 7.8 | 7.8 | 0.9 | 1.9 |
| pneumoniae | | | | | | |
| Pseud. | 15.6 | 15.6 | 3.9 | 15.6 | 0.9 | 3.9 |
| aeruginosa | | | | | | |
| Candida | Turbid | Growth | 15.6 | 15.6 | 3.9 | 3.9 |
| albicans | at neat | at neat | | | | |

### EXAMPLE 7

Surgihoney Emulsion

Preparation

10 g Surgihoney was dissolved in 10 ml of glycerol. 10 ml of paraffin oil was then added to a Rheometer (TA Instruments AR-G2) which had a Jacket Peltier and vane geometry attached. 1 ml of PGPR (Polyglycerol polyricinoleate) was then added. The rheometer was then started under the following conditions; Shear rate 2000 1/s, Temperature set at 37.5° C. After 2 minutes, 10 ml of Surgihoney-glycerol solution was added dropwise. Once a total of 10 minutes had elapsed the emulsion was transferred from the Jacket Peltier to a container.

Optical Microscopy

Optical microscopy revealed that the emulsion contained reverse micelles which encapsulated Surgihoney. Such micelles can be observed in FIG. 4. The average micelle diameter was found to be 178 μm.

Hydrogen Peroxide Tests

Hydrogen peroxide stick tests (Purchased from Sigma Aldrich (Quantofix®)) were used to detect hydrogen peroxide in the emulsion. The tests were carried out before and after addition of water, and showed that before addition of water, the emulsion produced no hydrogen peroxide, and after water was added, the emulsion tested positive for hydrogen peroxide. A positive test was indicated by a colour change to blue.

Stability Test

The emulsion maintained its capacity to generate hydrogen peroxide after storage at ambient conditions for at least four weeks.

Spray Test

The emulsion was added to a pump-action spray bottle and was found to be sprayable.

### EXAMPLE 8

Effects of Different Parameters on Stability of Surgihoney Emulsions

The effects of changing the Surgihoney emulsion preparation method described in Example 7, one parameter at a time, were investigated. The changes and their effects are summarised below.

i) Proportion of the Oil Phase to the Surgihoney-Glycerol Phase

Oil volumes greater than 10 ml, and less than 10 ml, were tested. The emulsion was found to be more stable when a lower volume of oil was used compared to the volume of the Surgihoney-glycerol phase. When the volume of the oil was less than 6 ml, the emulsion was found to separate by less than 3% in total volume over 72 hours. A volume of 4 ml allowed a separation of just 1.3% of the total volume over 72 hours. This stability is far greater than that of the method described in Example 7, which provided an emulsion with a separation of 9.4% of total volume over the same time period.

ii) Volume of PGPR

PGPR volumes up to 4 ml, and less than 1 ml, were tested. The emulsion was more stable when a higher amount of PGPR was used. At a volume of 4 ml, PGPR provided greater stability than with use of lower volumes, and far greater stability than that of the emulsion described in Example 7.

iii) Shear Rate

Shear rates from 1000 1/s to 3000 1/s were tested. The emulsion was more stable when a higher shear rate was applied. A shear rate of 3000 1/s produced the most stable emulsion. Separation of only 4.6% of the total volume over 72 hours was observed for emulsion prepared at this shear rate, compared with a separation volume of 9.4% of the total volume over the same time period for emulsion prepared as described in Example 7.

iv) Temperature

Temperatures from 20° C. to 40° C. were tested. There was no noticeable trend regard the stability of the emulsions as temperature was increased. However a temperature of 40° C. produced the most stable emulsion. Separation of only 3.1% of the total volume over 72 hours was observed for this emulsion.

v) Length of Shear

Shear times of 20 minutes and 30 minutes were tested, in addition to that used in the preparation method described in Example 7. However, there was no significant difference produced by extending the shear time.

vi) Order of Reagent Addition

The effect of changing the order in which the reagents are added to the rheometer was tested. The effect of adding all of the components before starting the rheometer was compared with the effect of adding the Surgihoney-glycerol and oil components first, then adding the PGPR after 1-2 minutes. The most stable emulsion was formed when the PGPR was added last. The resulting emulsion provided a separation volume of 2.8% of the total volume over 120 hours.

vii) Concentration of Surgihoney Dissolved in Glycerol

The following ratios of Surgihoney (g) to glycerol (ml) were tested: 1 g:1 ml; 0.5 g:1 ml; 2 g:1 ml. The ratio that produced the most stable emulsion was 1 g:1 ml, the same ratio used in the preparation method described in Example 7.

viii) Sodium Chloride

When sodium chloride is dissolved in the polar layer of the emulsion, it increases the polarity of this layer. It also forms electrostatic interactions with the lipid layer of the emulsion. The electrostatic interactions and increased polarity could improve stability and reduce coalescence. However, addition of sodium chloride (1 g, 2 g or 4 g) was not found to influence the stability of the emulsion.

The effects of the changes are summarised in the table below:

TABLE 8

| | | | | | | | | Stability |
|---|---|---|---|---|---|---|---|---|
| | SH | Glycerol | Paraffin | PGPR | Shear | Temp. | Order of | (% total vol. |
| Emulsion | (g) | (ml) | Oil (ml) | (ml) | rate (1/s) | (° C.) | addition | after 72 hrs) |
| Ex 7 | 10 | 10 | 10 | 1 | 2000 | 37.5 | Oil, then | 9.4 |
| | | | | | | | PGPR, then | |
| | | | | | | | SH/glycerol | |
| Ex 8 (i) | 10 | 10 | 6 | 1 | 2000 | 37.5 | Oil, then | 2.8 |
| | | | | | | | PGPR, then | |
| | | | | | | | SH/glycerol | |
| Ex 8 (i) | 10 | 10 | 4 | 1 | 2000 | 37.5 | Oil, then | 1.3 |
| | | | | | | | PGPR, then | |
| | | | | | | | SH/glycerol | |
| Ex 8 (ii) | 10 | 10 | 10 | 4 | 2000 | 37.5 | Oil, then | 2.7 |
| | | | | | | | PGPR, then | |
| | | | | | | | SH/glycerol | |
| Ex 8 (iii) | 10 | 10 | 10 | 1 | 3000 | 37.5 | Oil, then | 4.6 |
| | | | | | | | PGPR, then | |
| | | | | | | | SH/glycerol | |
| Ex 8 (iv) | 10 | 10 | 10 | 1 | 2000 | 40.0 | Oil, then | 3.1 |
| | | | | | | | PGPR, then | |
| | | | | | | | SH/glycerol | |
| Ex 8 (vi) | 10 | 10 | 10 | 1 | 2000 | 37.5 | SH/glycerol | 2.8* |
| | | | | | | | and oil, then | |
| | | | | | | | PGPR | |

*(after 120 hrs)

### EXAMPLE 9

Surgihoney Emulsions with High Stability

The results from the changes described in Example 8 were used to design a further method of preparing Surgihoney emulsion. This method is described below.

Preparation 10 g Surgihoney was dissolved in 10 ml of glycerol. 4, 6, 8, or 10 ml of Paraffin oil was then added to the rheometer (TA Instruments AR-G2) which had a Jacket Peltier and vane geometry attached. 10 ml of Surgihoney-glycerol solution was then added to the rheometer. The rheometer was then started under the following conditions; Shear rate 3000 1/s, Temperature 40° C., gap 4000 μm, Run time 10 minutes. After 1 minute 4 ml of PGPR (Polyglycerol polyricinoleate) was then added. Once a total of 10 minutes had elapsed the emulsion was transferred from the rheometer to a container.

TABLE 9

| | | | | | Total |
|---|---|---|---|---|---|
| | | | | Total Volume | Volume |
| | Surgihoney- | | | Separation | Separation |
| Formulation | Glycerol (ratio- | | Paraffin oil | <11 Days | after 20 |
| number | 1 g:1 ml) (ml) | PGPR (ml) | (ml) | (%) | days (%) |
| 1 | 10 | 4 | 10 | 0 | 0.7 |
| 2 | 10 | 4 | 4 | 0 | 0.9 |
| 3 | 10 | 4 | 4 | 0 | 1.0 |
| 4 | 10 | 4 | 4 | 0 | 1.6 |
| 5 | 10 | 4 | 6 | 0 | 1.2 |
| 6 | 10 | 4 | 8 | 0 | 0.9 |

All of the formulations were found to be highly stable, with a slight increase in stability observed as the volume of paraffin oil used was increased.

### EXAMPLE 10

Surgihoney Cream Formulation

1.5 g of Surgihoney was dissolved in 1.5 ml of glycerol. 1 g of sodium alginate was then dissolved in the Surgihoney-glycerol solution. Next 10 ml of Paraffin oil was added to the Rheometer (TA Instruments AR-G2) which had a Jacket Peltier and vane geometry attached. 1 ml of PGPR (Polyglycerol polyricinoleate) was then added. The rheometer was then started under the following conditions; Shear rate 2000 1/s, Temperature set at 37.5° C., gap 4000 μm, Run time 10 minutes. After 2 minutes, 1.5 ml of the Surgihoney-alginate and glycerol solution was added to the rheometer. After 3 minutes 8 ml of calcium chloride solution was added dropwise to the rheometer. Once a total of 10 minutes had elapsed the emulsion was transferred from the Jacket Peltier to a container.

### EXAMPLE 11

Non-Aqueous Surgihoney Cream Formulation

The method described in Example 10 uses water to dissociate calcium chloride into its ions. This could potentially activate the Surgihoney to produce hydrogen peroxide, and limit the stability of the cream formulation. However, we have appreciated that calcium chloride can be dissociated using non-aqueous solvents, such as ethanol or acetic acid. We have also appreciated that glycerol is able to bind to free water. This property allows water to be used to dissolve the alginate, provided sufficient glycerol is present to prevent premature release of hydrogen peroxide.

The method described below uses ethanol as a solvent for calcium chloride, and glycerol to bind free water in the alginate solution.

1 g of sodium alginate is dissolved in 15 ml water. Next 30 ml glycerol is added to the alginate solution and mixed. Then 30 g Surgihoney is then dissolved in the solution. 10 ml of Paraffin oil is then added to the rheometer (TA Instruments AR-G2) which has a Jacket Peltier and vane geometry attached. 10 ml of Surgihoney solution is then added to the rheometer. The rheometer is then started under the following conditions: Shear rate 3000 1/s, Temperature 40° C., gap 4000 μm, Run time 10 minutes. After 1 minute 4 ml of PGPR (Polyglycerol polyricinoleate) is added. After 2 minutes 8 ml of non-aqueous calcium chloride solution (1M Calcium chloride in ethanol) is added dropwise to the rheometer. Once a total of 10 minutes has elapsed, the emulsion is transferred from the rheometer to a container.

Summary of emulsion formulations in Examples 7-11:

TABLE 10

| Emulsion/ | SH | Glycerol | Paraffin | PGPR | NaAlg(g)/ | | Shear | Temp. | |
|---|---|---|---|---|---|---|---|---|---|
| cream | (g) | (ml) | Oil (ml) | (ml) | CaCl₂(ml) | | rate (1/s) | (° C.) | Order of addition |
| Ex 7 | 10 | 10 | 10 | 1 | — | | 2000 | 37.5 | Oil, then PGPR, |
| | | | | | | | | | then SH/glycerol |
| Ex 9 | 10 | 10 | 4, 6, 8, | 4 | — | | 3000 | 40.0 | Oil and SH/ |
| | | | or 10 | | | | | | glycerol, then |
| | | | | | | | | | PGPR |
| Ex 10 | 1.5 | 1.5 | 10 | 1 | 1/8 | (aq) | 2000 | 37.5 | Oil and PGPR, |
| | | | | | | | | | then SH/glycerol/ |
| | | | | | | | | | NaAlg, then CaCl₂ |
| Ex 11 | 30 | 30 | 10 | 4 | 1/8 | (non-aq) | 3000 | 40 | Oil and SH/ |
| | | | | | | | | | glycerol/NaAlg(aq), |
| | | | | | | | | | then PGPR, then |
| | | | | | | | | | CaCl₂ (non-aq) |

1. A composition for generating antimicrobial activity, which comprises: a lipophilic phase; an aqueous phase; an enzyme that is able to convert a substrate to release hydrogen peroxide; and a substance that includes a substrate for the enzyme.

2. A composition according to claim 1, which is in the form of colloid or a suspension.

3. A composition according to claim 1 or 2, which is in the form of an emulsion.

4. A composition according to any preceding claim, wherein the lipophilic phase comprises an oil or a wax.

5. A composition according to claim 4, wherein the oil or wax is selected from:
Aleurites Moluccana Seed Oil; Grape (Vitis Vinifera) Seed Oil; Almond Oil NF; Hybrid Safflower (Carthamus Tinctorius) Oil; Anhydrous Lanolin USP; Isopropyl Myristate; Apricot Kernel Oil; Isopropyl Palmitate; Avocado (Persea Gratissima) Oil; Jojoba (Buxus Chinensis) Oil; Babassu Oil; Lanolin; Beeswax; Macadamia (Ternifolia) Nut Oil; Borage (Borago Officinalis) Seed Oil; Mangifera Indica (Mango) Seed Butter; Brazil Nut Oil; Mineral Oil; C12-15 Alkyl Benzoate; Myristyl Myristate; Cannabis Sativa Seed Oil; Olive (Olea Europaea) Oil; Canola Oil; Oryza Sativa (Rice Bran) Oil; Caprylic/Capric Triglyceride; Peanut Oil NF; Carrot (Daucus Carota Sativa) Seed Oil; Petrolatum; Castor (Ricinus Communis) Oil; PPG-15 Stearyl Ether; Ceresin; Retinyl Palmitate; Cetearyl Alcohol; Safflower (Carthamus Tinctorius) Oil; Cetyl Alcohol; Sesame (Sesamum Indicum) Oil; Cetyl Esters; Shea Butter (Butyrospermum Parkii); Cetyl Palmitate; Soybean (Glycine Soja) Oil; Coconut Oil; Stearic Acid; Daucus Carota Sativa (Carrot) Root Extract; Stearyl Alcohol; Diisopropyl Adipate; Sunflower (Helianthus Annus) Oil; Dimethicone; Sweet Almond (Prunus Amygdalus Dulcis) Oil; Dog Rose (Rosa Canina) Hips Oil; Theobroma Cacao (Cocoa) Seed Butter; Emu Oil; Tocopherol; Evening Primrose Oil.

6. A composition according to claim 4 or 5, wherein the oil or wax is a beeswax.

7. A composition according to any preceding claim, which further comprises one or more emulsifying agents.

8. A composition according to claim 7, wherein the emulsifying agent has an hydrophilic-lipophilic balance (HLB) value, or the emulsifying agents have a combined HLB value, in the range 3-6 or 8-18.

9. A composition according to claim 8, wherein the emulsifying agent is selected from:
Calcium Stearoyl Lactylate; Ceteareth-20; Cetearyl Glucoside; Ceteth-10; Ceteth-2; Ceteth-20; Cocamide MEA; Glyceryl Laurate; Glyceryl Stearate; Glyceryl Stearate (and) PEG-100 Stearate; Glyceryl Stearate SE; Glycol Distearate; Glycol Stearate; Isoceteth-20; Isosteareth-20; Lauramide DEA; Laureth-23; Laureth-4; Lecithin; Linoleamide DEA; Methyl Glucose Sesquistearate; Oleth-10; Oleth-10/Polyoxyl 10 Oleyl Ether NF; Oleth-2; Oleth-20; Oleth-20; PEG-100 Stearate; PEG-20 Almond Glycerides; PEG-20 Methyl Glucose Sesquistearate; PEG-25 Hydrogenated Castor Oil; PEG-30 Dipolyhydroxystearate; PEG-4 Dilaurate; PEG-40 Sorbitan Peroleate; PEG-60 Almond Glycerides; PEG-8 Laurate; PEG-80 Sorbitan Laurate; Polysorbate 20; Polysorbate 60; Polysorbate 80; Polysorbate 85; Sodium Stearoyl Lactylate; Sorbitan Isostearate; Sorbitan Laurate; Sorbitan Oleate; Sorbitan Sesquioleate; Sorbitan Stearate; Sorbitan Stearate (and) Sucrose Cocoate; Sorbitan Trioleate; Stearamide MEA; Steareth-2; Steareth-21.

10. A composition according to any of claims 7 to 9, wherein the emulsifying agent is a lecithin.

11. A composition according to any preceding claim which is formulated for topical application.

12. A composition according to any preceding claim which is in the form of a cream or a lotion.

13. A composition according to any preceding claim, wherein the enzyme is additional to any enzyme activity able to convert the substrate to release hydrogen peroxide that may be present in the substance.

14. A composition according to any preceding claim, wherein the composition is a storage-stable composition that does not include sufficient free water to allow the enzyme to convert the substrate.

15. A composition according to any preceding claim, wherein the enzyme is a purified enzyme.

16. A composition according to any preceding claim, wherein the enzyme is an oxidoreductase enzyme.

17. A composition according to claim 16, wherein the oxidoreductase enzyme is a glucose oxidase.

18. A composition according to any preceding claim, wherein the substance includes a purified substrate for the enzyme.

19. A composition according to claim 18, wherein the purified substrate comprises a purified sugar.

20. A composition according to claim 19, wherein the purified sugar comprises D-glucose, hexose, or D-galactose.

21. A composition according to any of claims 1 to 17, wherein the substance is an unrefined natural substance.

22. A composition according to claim 21, wherein the unrefined natural substance lacks catalase activity.

23. A composition according to claim 21 or 22, wherein the unrefined natural substance is an unrefined natural sugar substance.

24. A composition according to claim 23, wherein the unrefined natural sugar substance is a honey.

25. A composition according to claim 24, wherein the honey is an unpasteurised honey, preferably a creamed unpasteurised honey.

26. A composition according to any preceding claim, which does not include any detectable hydrogen peroxide.

27. A composition according to any preceding claim, wherein the composition is a sterile composition.

28. A composition according to any preceding claim, wherein the ratio of the lipophilic phase to the aqueous phase is from 9:1 to 1:9, 8:1 to 1:8, 7:1 to 1:7, 6:1 to 1:6, 5:1 to 1:5, 4:1 to 1:4, 3:1 to 1:3, or 2:1 to 1:2 (v/v).

29. A composition according to any preceding claim, which comprises 5-95%, 10-95%, 15-95%, 20-95%, 25-95%, 30-95%, 35-95%, 40-95%, 45-95%, 50-95%, 55-95%, 60-95%, 65-95%, 70-95%, 75-95%, 80-95%, 85-95%, or 90-95% (v/v) lipophilic phase (including any emulsifying agent present).

30. A composition according to any of claims 1 to 28, which comprises 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, or 5-10% (v/v) lipophilic phase (including any emulsifying agent present).

31. A composition according to any preceding claim, which comprises 5-95%, 10-95%, 15-95%, 20-95%, 25-95%, 30-95%, 35-95%, 40-95%, 45-95%, 50-95%, 55-95%, 60-95%, 65-95%, 70-95%, 75-95%, 80-95%, 85-95%, or 90-95% (v/v) aqueous phase.

32. A composition according to any of claims 1 to 31, which comprises 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, or 5-10% (v/v) aqueous phase.

33. A composition according to any preceding claim, which comprises 1-60%, 1-50%, 1-40%, 1-30%, 1-20%, or 1-10% (w/v) of the substance.

34. A composition according to any of claims 1 to 32, which comprises 1-60%, 5-60%, 10-60%, 15-60%, 20-60%, 25-60%, 30-60%, 35-60%, 40-60%, 45-60%, or 50-60% (w/v) of the substance.

35. A composition according to any preceding claim, which comprises less than 20% water, preferably 10-19% water.

36. A composition according to any preceding claim, which comprises 1-1500 units, 15-1500 units, 30-1500 units, 50-1500 units, 100-1500 units, 1-<685 units, 15-<685 units, 30-<685 units, 50-<685 units, 100-<685 units, 500-1000 units, 685-1000 units, or 100-500 units, of the enzyme, preferably glucose oxidase, per gram of the composition.

37. A composition according to any preceding claim, which comprises sufficient enzyme and substrate to provide for sustained release of 0.1 to less than 2 mmol/litre hydrogen peroxide for a period of at least 24 hours.

38. A composition according to any preceding claim for use as a medicament.

39. A composition according to any of claims 1 to 37 for use in the prevention or treatment of a microbial infection.

40. Use of a composition according to any of claims 1 to 37 in the manufacture of a medicament for the prevention or treatment of a microbial infection.

41. Use according to claim 39 or 40, wherein the prevention or treatment is by topical administration of the composition.

42. A method of prevention or treatment of a microbial infection, which comprises administering an effective amount of a composition according to any of claims 1 to 37 to a subject in need of such treatment.

43. A method according to claim 42, wherein the composition is administered topically to the subject.

44. Use according to any of claims 39 to 41, or a method according to claim 42 or 43, wherein the microbial infection is a viral infection, preferably a herpes simplex virus (HSV) infection.

45. Use according to any of claims 39 to 41, or a method according to claim 42 or 43, wherein the microbial infection is a fungal infection.

46. Use according to any of claims 39 to 41, or a method according to claim 42 or 43, wherein the microbial infection is a bacterial infection.

47. A method of making a composition according to any of claims 1 to 37, which comprises mixing a lipophilic component, an aqueous component, an enzyme that is able to convert a substrate to release hydrogen peroxide, and a substance that includes a substrate for the enzyme to form the composition.

48. A composition for generating antimicrobial activity, comprising: a first phase; a second phase; an enzyme that is able to convert a substrate to release hydrogen peroxide; and a substance that includes a substrate for the enzyme, wherein the first phase and the second phase are immiscible.

49. A composition according to claim 48, wherein the first phase is less polar than the second phase.

50. A composition according to claim 48 or claim 49, wherein the first phase is a non-polar phase, and the second phase is a polar phase.

51. A composition according to any of claims 48 to 50, wherein the first phase is a lipophilic phase and the second phase is an aqueous phase.

52. A composition according to any of claims 48 to 51, wherein the second phase comprises a non-aqueous solvent, optionally glycerol, dimethylsulphoxide, propylene glycol or polyethylene glycol.

53. A composition according to any of claims 48 to 52, wherein the first phase is, or comprises, an oil, optionally olive oil, corn oil, vegetable oil, sunflower oil or paraffin oil.

54. A composition according to any of claims 48 to 53, comprising an emulsifier, preferably a surfactant, optionally wherein the surfactant is, or comprises, TWEEN, SPAN, Poloxamer or Polyglycerol polyricinoleate.

55. A composition for generating antimicrobial activity, comprising: an oil; an emulsifier; an enzyme that is able to convert a substrate to release hydrogen peroxide; and a substance that includes a substrate for the enzyme.

56. A composition according to claim 55, comprising a non-aqueous solvent.

57. A composition according to claim 56, wherein the non-aqueous solvent is a polar solvent.

58. A composition according to claim 56 or claim 57, wherein the non-aqueous solvent is an organic solvent.

59. A composition according to any of claims 56 to 58, wherein the non-aqueous solvent is, or comprises, glycerol, dimethylsulphoxide, propylene glycol or polyethylene glycol.

60. A composition according to any of claims 55 to 59, wherein the oil is, or comprises, olive oil, corn oil, vegetable oil, sunflower oil or paraffin oil.

61. A composition according to any of claims 55 to 60, wherein the emulsifier is, or comprises, a surfactant.

62. A composition according to claim 61, wherein the surfactant is, or comprises, TWEEN, SPAN, Poloxamer or Polyglycerol polyricinoleate.

63. A composition according to any of claims 48 to 62, wherein the enzyme is additional to any enzyme activity able to convert the substrate to release hydrogen peroxide that may be present in the substance.

64. A composition according to any of claims 48 to 63, which does not comprise sufficient free water to allow the enzyme to convert the substrate.

65. A composition according to any of claims 48 to 64 which is storage stable.

66. A composition according to any of claims 48 to 65, which provides for sustained release of hydrogen peroxide at a level of less than 2 mmol/litre for a period of at least twenty four hours, following dilution of the composition.

67. A composition according to any of claims 48 to 66, which provides for sustained release of at least 0.1, 0.5, 1 or 1.5 mmol/litre hydrogen peroxide for a period of at least 24 hours.

68. A composition according to any of claims 48 to 67, wherein the enzyme is a purified enzyme.

69. A composition according to any of claims 48 to 68, wherein the enzyme is an oxidoreductase enzyme, preferably glucose oxidase.

70. A composition according to any of claims 48 to 69, wherein the substance lacks catalase activity.

71. A composition according to any of claims 48 to 70, wherein the substance is an unrefined substance, such as an unrefined natural substance.

72. A composition according to any of claims 48 to 71, wherein the substance is a sugar substance, preferably comprising glucose.

73. A composition according to any of claims 48 to 72, wherein the substance is, or comprises, honey.

74. A composition according to any of claims 48 to 70, wherein the substance includes a purified substrate for the enzyme, preferably a purified sugar such as purified glucose.

75. A composition according to any of claims 48 to 74, comprising 10-60%, preferably 20-50%, more preferably 35-45% (w/w) non-aqueous solvent.

76. A composition according to any of claims 48 to 75, comprising 10-40%, preferably 20-30% (w/w) oil.

77. A composition according to any of claims 48 to 76, comprising 1-10%, preferably 1-5% (w/w) emulsifier

78. A composition according to any of claims 48 to 77, comprising 10-50%, preferably, more preferably 20-40% (w/w) substance which comprises a substrate for the enzyme.

79. A composition according to any of claims 48 to 74, comprising 20-50% (w/w) non-aqueous solvent, 20-30% (w/w) oil, 1-5% (w/w) emulsifier and 20-40% (w/w) substance which comprises a substrate for the enzyme.

80. A composition according to any of claims 48 to 74, comprising 10-60% (w/w) non-aqueous solvent, 10-40% (w/w) oil, 1-10% (w/w) emulsifier and 10-50% (w/w) substance which comprises a substrate for the enzyme.

81. A composition according to any of claims 48 to 74, comprising 35-45% (w/w) non-aqueous solvent, 20-30% (w/w) oil, 1-5% (w/w) emulsifier and 25-35% (w/w) substance which comprises a substrate for the enzyme.

82. A composition according to any of claims 48 to 74, wherein the ratio of the first phase to the second phase is ≤1:1 (v/v).

83. A composition according to claim 82, wherein the ratio of the first phase to the second phase is <0.6:1 (v/v).

84. A composition according to claim 82, wherein the ratio of the first phase to the second phase is 0.4:1 (v/v).

85. A composition according to any of claims 48 to 74, or 82 to 84, wherein the first phase is present at less than 60% (v/v) of the composition.

86. A composition according to claim 85, wherein the first phase is present at 10% to less than 60% (v/v) of the composition.

87. A composition according to claim 85, wherein the first phase is present at 10% to less than 40% (v/v) of the composition.

88. A composition according to claim 85, wherein the first phase is present at 10% to less than 30% (v/v) of the composition.

89. A composition according to claim 85, wherein the first phase is present at 10% to less than 25% (v/v) of the composition.

90. A composition according to any of claims 48 to 74, or 82 to 89, which comprises an emulsifier, wherein the emulsifier is present at up to 25% (v/v) of the composition.

91. A composition according to claim 90, wherein the emulsifier is present at 1-25% (v/v) of the composition.

92. A composition according to claim 90, wherein the emulsifier is present at 5-25% (v/v) of the composition.

93. A composition according to claim 90, wherein the emulsifier is present at 10-25% (v/v) of the composition.

94. A composition according to any of claims 48 to 74, or 82 to 93, wherein the ratio of the amount of the substance that includes a substrate for the enzyme to the volume of the second phase is from 0.5:1 to 2:1.

95. A composition according to claim 94, wherein the ratio of the amount of the substance that includes a substrate for the enzyme to the volume of the second phase is 1:1.

96. A composition according to any of claims 48 to 74, or 82 to 95, wherein the amount of the substance that includes a substrate for the enzyme in the composition is up to 70% (w/v) of the composition.

97. A composition according to claim 96, wherein the amount of the substance that includes a substrate for the enzyme in the composition is 5-70% (w/v) of the composition.

98. A composition according to claim 96, wherein the amount of the substance that includes a substrate for the enzyme in the composition is 10-70% (w/v) of the composition.

99. A composition according to claim 96, wherein the amount of the substance that includes a substrate for the enzyme in the composition is 20-70% (w/v) of the composition.

100. A composition according to claim 96, wherein the amount of the substance that includes a substrate for the enzyme in the composition is 30-70% (w/v) of the composition.

101. A composition according to any of claims 48 to 100, which is an emulsion, preferably wherein the emulsion comprises reverse micelles.

102. A composition according to claim 101, wherein the reverse micelles are formed by the second phase.

103. A composition according to any of claims 48 to 102, wherein the enzyme and the substance that includes a substrate for the enzyme is dissolved in the second phase.

104. A composition according to any of claims 48 to 103, wherein the first phase is, or comprises paraffin oil.

105. A composition according to any of claims 48 to 104, wherein the second phase is, or comprises glycerol.

106. A composition according to any of claims 48 to 105, wherein the emulsifier is, or comprises Polyglycerol polyricinoleate (PGPR).

107. A composition according to any of claims 48 to 106, wherein the enzyme that is able to convert a substrate to release hydrogen peroxide is, or comprises purified glucose oxidase, and the substance that includes a substrate for the enzyme is, or comprises honey.

108. A composition according to any of claims 48 to 106, wherein the enzyme that is able to convert a substrate to release hydrogen peroxide is, or comprises purified glucose oxidase, and the substance that includes a substrate for the enzyme is, or comprises purified glucose.

109. A composition according to any of claims 48 to 108, which is a cream.

110. A composition according to any of claims 48 to 109, which further comprises a viscosity-increasing agent.

111. A composition according to claim 110, wherein the viscosity-increasing agent is, or comprises a hydrocolloid.

112. A composition according to claim 111, wherein the hydrocolloid is, or comprises a polysaccharide.

113. A composition according to claim 111 or 112, wherein the hydrocolloid is, or comprises a hydrocolloid thickener.

114. A composition according to claim 113, wherein the hydrocolloid thickener is starch, modified starch, xanthan, a galactomannan (such as guar gum, locust bean gum, and tara gum), gum Arabic or acacia gum, gum karaya, gum tragacanth, konjac maanan, or a cellulose derivative, such as carboxymethyl cellulose, methyl cellulose, or hydroxypropylmethyl cellulose.

115. A composition according to claim 111 or 112, wherein the hydrocolloid is, or comprises a cross-linked hydrocolloid.

116. A composition according to claim 115, wherein the cross-linked hydrocolloid is a cross-linked polysaccharide.

117. A composition according to claim 116, wherein the cross-linked polysaccharide is cross-linked alginate, pectin, carrageenan, gelatin, gellan, agar, agarose, modified starch, or a cellulose derivative, such as methyl cellulose or hydroxypropylmethyl cellulose.

118. A composition according to claim 115 or 116, wherein molecules of the hydrocolloid are crosslinked by cations.

119. A composition according to claim 118, wherein the hydrocolloid is alginate, carrageenan or pectin.

120. A composition according to claim 119, wherein the hydrocolloid is alginate cross-linked by calcium ions.

121. A method of making a composition, comprising mixing a first component, a second component, an enzyme that is able to convert a substrate to release hydrogen peroxide, and a substance that includes a substrate for the enzyme to form the composition, wherein the first component and second component are immiscible.

122. A method of making a composition, comprising mixing an oil, an enzyme that is able to convert a substrate to release hydrogen peroxide, and a substance that includes a substrate for the enzyme to form the composition.

123. The method according to claim 121 or 122 comprising mixing a non-aqueous solvent.

124. A method of making a composition according to any of claims 48 to 120, which comprises mixing the enzyme, the substance that includes a substrate for the enzyme, liquid of the second phase, liquid of the first phase, and optionally an emulsifier, under a high rate of shear for sufficient time to form an emulsion.

125. A method according to claim 124, wherein the enzyme, the substance that includes a substrate for the enzyme, liquid of the second phase, and liquid of the first phase are pre-mixed under a high rate of shear before contacting the pre-mixed ingredients with the emulsifier and mixing of the mixture comprising the pre-mixed ingredients and the emulsifier under the high rate of shear.

126. A method according to claim 124 or 125, wherein the enzyme and the substance that includes a substrate for the enzyme are dissolved in the liquid of the second phase to form a solution before contacting the solution with the liquid of the first phase.

127. A method according to any of claims 124 to 126, wherein the high rate of shear is 1000 1/s to 4000 1/s.

128. A method according to any of claims 124 to 127, wherein the high rate of shear is >2500 1/s to 3500 1/s.

129. A method according to any of claims 124 to 128, wherein the mixing is carried out at 20° C. to 40° C.

130. A method according to any of claims 124 to 129, wherein the mixing is carried out at 35° C. to 40° C.

131. A method according to any of claims 124 to 130, wherein the mixing is carried out at 38° C. to 40° C.

132. A method according to any of claims 124 to 131, wherein the enzyme, the substance that includes a substrate for the enzyme, liquid of the second phase, liquid of the first phase, and the emulsifier (if present), are mixed under a high rate of shear for at least 5 minutes.

133. A method according to any of claims 124 to 132, wherein the enzyme, the substance that includes a substrate for the enzyme, liquid of the second phase, liquid of the first phase, and the emulsifier (if present), are mixed under a high rate of shear for 5 to 30 minutes.

134. A method according to any of claims 124 to 133, which further comprises mixing a viscosity-increasing agent with the enzyme, the substance that includes a substrate for the enzyme, liquid of the second phase, liquid of the first phase, and the emulsifier (if present), under the high shear rate to form a cream.

135. A method according to claim 134, wherein the viscosity-increasing agent is, or comprises a hydrocolloid.

136. A method according to claim 135, wherein the hydrocolloid is, or comprises a polysaccharide.

137. A method according to claim135 or 136, wherein the hydrocolloid is, or comprises a hydrocolloid thickener.

138. A method according to claim 137, wherein the hydrocolloid thickener is starch, modified starch, xanthan, a galactomannan (such as guar gum, locust bean gum, and tara gum), gum Arabic or acacia gum, gum karaya, gum tragacanth, konjac maanan, or a cellulose derivative, such as carboxymethyl cellulose, methyl cellulose, or hydroxypropylmethyl cellulose.

139. A method according to claim 135 or 136, wherein the hydrocolloid is, or comprises a hydrocolloid gelling agent.

140. A method according to claim 139, wherein the hydrocolloid gelling agent comprises alginate, pectin, carrageenan, gelatin, gellan, agar, agarose, modified starch, or a cellulose derivative, such as methyl cellulose or hydroxypropylmethyl cellulose.

141. A method according to claim 139 or 140, wherein the hydrocolloid gelling agent is capable of forming a gel by ionotropic gelation in the presence of cations, and wherein the method further comprises mixing the cations with the hydrocolloid gelling agent, the enzyme, the substance that includes a substrate for the enzyme, liquid of the second phase, liquid of the first phase, and the emulsifier (if present), under the high shear rate to form the cream.

142. A method according to claim 141, wherein the hydrocolloid gelling agent, the enzyme, the substance that includes a substrate for the enzyme, liquid of the second phase, liquid of the first phase, and the emulsifier (if present), are mixed to form a mixture prior to contacting the cations with the mixture.

143. A method according to claim 142, wherein the cations are provided in non-aqueous solution, such as ethanol or acetic acid.

144. A method according to any of claims 141 to 143, wherein the cations are, or comprise calcium ions.

145. A method according to any of claims 141 to 144, wherein the hydrocolloid gelling agent is contacted with the liquid of the second phase, the enzyme, and the substance that includes a substrate for the enzyme, prior to contact with the liquid of the first phase.

146. A method according to any of claims 141 to 145, wherein the hydrocolloid gelling agent is, or comprises alginate, carrageenan or pectin.

147. A method according to claim 146, wherein the hydrocolloid gelling agent is, or comprises alginate.

148. A method according to any of claims 141 to 147, wherein the hydrocolloid gelling agent is provided in aqueous solution, and the second phase is glycerol, wherein the glycerol is present in sufficient amount to bind free water in the composition and thereby prevent the enzyme catalysing release of hydrogen peroxide from the substance that includes a substrate for the enzyme.

149. A pharmaceutical composition comprising a composition according to any of claims 48 to 120, and a pharmaceutically acceptable carrier excipient or diluent.

150. A composition according to any of claims 48 to 120, or a pharmaceutical composition according to claim 149, for use as a medicament.

151. A composition according to any of claims 48 to 120, or a pharmaceutical composition according to claim 149, for use in the prevention or treatment of microbial infection.

152. Use of a composition according to any of claims 48 to 120, or a pharmaceutical composition according to claim 149, in the manufacture of a medicament for the prevention or treatment of microbial infection.

153. A method of preventing or treating a microbial infection which comprises administering an effective amount of a composition according to any of claims 48 to 120, or a pharmaceutical composition according to claim 149, to a subject in need of such a treatment.

154. A composition according to claim 151, use according to claim 152, or a method according to claim 153, wherein the microbial infection is: a nasal infection, such as sinusitis or rhinosinusitis; a respiratory tract infection, such as an upper respiratory tract infection (e.g. tonsillitis, laryngitis or sinusitis) or a lower respiratory tract infection (e.g. bronchitis, pneumonia, bronchiolitis or tuberculosis); an infection associated with chronic obstructive pulmonary disease (COPD), cystic fibrosis, bronchiectasis, asthma, or an HIV/AIDS-associated respiratory infection, or a respiratory infection associated with terminal disease.
